(19) United States
(12) Patent Application Publication
(10) Pub. No.: US 2026/0049333 A1
(43) Pub. Date: Feb. 19, 2026

(54) Variant AAV and Compositions, Methods and Uses for Gene Transfer to Cells, Organs and Tissues

(71) Applicant: The Children's Hospital of Philadelphia, Philadelphia, PA (US)

(72) Inventors: Katherine A. High, Merion Station, PA (US); Mustafa N. Yazicioglu, Broomall, PA (US); Xavier Anguela, Barcelona (ES)

(73) Assignee: The Children's Hospital of Philadelphia, Philadelphia, PA (US)

(21) Appl. No.: 19/174,833

(22) Filed: Apr. 9, 2025

Related U.S. Application Data
(63) Continuation of application No. 15/834,011, filed on Dec. 6, 2017, now abandoned.
Application No. 15/834,011 is a continuation of application No. 14/338,200, filed on Jul. 22, 2014, now Pat. No. 9,840,719.
(60) Provisional application No. 61/857,161, filed on Jul. 22, 2013.
(60) Provisional application No. 61/985,365, filed on Apr. 28, 2014.

(51) Int. Cl. C12N 15/86; A61K 38/00; A61K 48/00; C07K 14/005; C12N 9/64

(52) U.S. Cl. CPC C12N 15/86; C07K 14/005; C12N 9/6408; C12Y 304/21022; A61K 38/00; A61K 48/0058; C12N 2750/14122; C12N 2750/14143; C12N 2830/008

(57) ABSTRACT

The invention relates to adeno-associated virus (AAV) serotype AAV-Rh74 and related AAV vectors, and AAV-Rh74 and related AAV vector mediated gene transfer methods and uses. In particular, AAV-Rh74 and related AAV vectors target polynucleotides to cells, tissues or organs for expression (transcription) of genes encoding therapeutic proteins and peptides, and polynucleotides that function as or are transcribed into inhibitory nucleic acid sequences.

### CROSS-REFERENCE TO RELATED APPLICATIONS

This application is a continuation of application Ser. No. 15/834,011, filed Dec. 6, 2017, which is a continuation of application Ser. No. 14/338,200, filed Jul. 22, 2014, now U.S. Pat. No. 9,840,719, which claims the benefit of priority to provisional application No. 61/985,365, filed Apr. 28, 2014, and provisional application No. 61/857,161, filed Jul. 22, 2013, all of which applications are expressly incorporated herein by reference in their entirety.

### SEQUENCE LISTING

The contents of the file named “074659-0583508-SEQUENCE LISTING.xml,” which was created on Apr. 8, 2025, and is 36,770 bytes in size, are hereby incorporated by reference in their entirety.

### INTRODUCTION

Genetic disorders, caused by absence or a defect in a desirable gene (loss of function) or expression of an undesirable or defective gene or (gain of function) lead to a variety of diseases. One example of a loss of function genetic disorder is hemophilia, an inherited bleeding disorder caused by deficiency in either coagulation factor VIII (FVIII, hemophilia A) or factor IX (FIX, hemophilia B). One example of a gain of function genetic disorder is Huntington's disease, a disease caused by a pathologic “HTT” gene (encodes the huntingtin protein) that encodes a mutated protein that accumulates within and leads to gradual destruction of neurons, particularly in the basal ganglia and the cerebral cortex.

Current treatment for hemophilia consists in the intravenous administration of recombinant clotting factor either on demand, in case a bleeding occurs, or prophylactically. However, this therapeutic approach has several drawbacks such as the need for repeated infusions, the cost of the treatment, the risk of developing anti-therapeutic factor immune responses, and the risk of potentially fatal bleedings. These limitations have prompted the development of gene-based therapies for hemophilia. To this end, hemophilia is ideal for gene transfer based therapy as 1) the therapeutic window is very wide, as levels just above 1% of normal already can result in a change in phenotype from severe to moderate, and levels of 100% are not associated to any side effects; 2) tissue specific expression of the therapeutic transgene is not strictly required; and 3) there is a considerable experience in measuring the endpoints of therapeutic efficacy. Furthermore, liver expression of clotting factor has been demonstrated to induce immunological tolerance to the clotting factor itself, reducing the likelihood of potentially harmful immune responses against clotting factor.

Currently, adeno-associated virus (AAV) vectors are recognized as the gene transfer vectors of choice since they have the best safety and efficacy profile for the delivery of genes in vivo. Of the AAV serotypes isolated so far, AAV2 and AAV8 have been used to target the liver of humans affected by severe hemophilia B. Both vectors worked efficiently and in the case of AAV8 long-term expression of the therapeutic transgene was documented. Recent data in humans showed that targeting the liver with an AAV vector achieves long-term expression of the FIX transgene at therapeutic levels.

While these data are promising, the identification of AAV serotypes with high tropism for liver and low seroprevalence in humans (a natural host for wild type AAV) is fundamental for 1) achieving therapeutic levels of transgene expression in liver at the lowest vector dose possible to decrease risk of triggering anti-AAV capsid immune responses; and 2) alternate AAV serotypes with unique seroprevalence will allow to treat those patients populations otherwise not eligible for AAV gene transfer due to pre-existing humoral immunity to AAVs. The invention addresses these needs and provides additional benefits.

### SUMMARY

The invention provides adeno-associated virus (AAV) serotype AAV-Rh74 vector, and related AAV vectors and viral particles. Such vectors include AAV-Rh74 which target hepatocyte cells of the liver, among other cell types. As a vector for polynucleotide sequence delivery, AAV-Rh74 and related AAV vectors drive expression of the polynucleotide in cells. Polynucleotides that encode proteins, such as proteins for therapeutic applications are able to be expressed at therapeutic levels after administration.

Exemplary AAV-Rh74 and related AAV vectors include AAV-Rh74 variants. Particular capsid variants include an Rh74 capsid sequence with an amino acid substitution at any one of amino acid positions 195, 199, 201 or 202, of RH74 VP1 capsid sequence (SEQ ID NO:1). In particular aspects, the residues correspond to an A, V, P or N amino acid at any one of amino acid positions 195, 199, 201 or 202 of RH74 VP1 capsid sequence (SEQ ID NO: 1). In more particular aspects, the capsid sequence has an A residue at amino acid position 195; a V residue at amino acid positions 199, a P residue at amino acid position 201, or an N residue at amino acid position 202 of RH74 VP1 capsid sequence (SEQ ID NO:1). In further more particular aspects, the capsid sequence has any two, three or all four of the following: an A residue at amino acid position 195; a V residue at amino acid positions 199, a P residue at amino acid position 201, or an N residue at amino acid position 202 of RH74 VP1 capsid sequence (SEQ ID NO: 1).

Recombinant AAV particles of the invention include an AAV capsid sequence of any capsid variant such as RHM4-1 (SEQ ID NO:5), RHM15-1 (SEQ ID NO:6), RHM15-2 (SEQ ID NO:7), RHM15-3/RHM15-5 (SEQ ID NO:8), RHM15-4 (SEQ ID NO:9) or RHM15-6 (SEQ ID NO:10). In particular embodiments, a recombinant AAV particle encapsidates or packages a vector genome (e.g., viral vector such as an AAV vector genome). Such invention recombinant AAV particles include a viral (e.g., AAV) vector genome which also includes a heterologous polynucleotide sequence.

AAV-Rh74 and related AAV vector mediated polynucleotide transfer produced protein expression levels that were significantly higher than several other serotypes currently studied in preclinical and clinical settings. In particular, AAV-Rh74 targets polynucleotides to the liver with efficiency at least comparable or superior to the gold standard for liver transduction, AAV8, both in mice and in hemophilia B dogs. (see, e.g., FIGS. 1 and 2). AAV-Rh74 variants such as, for example, capsid variant RHM4-1 (SEQ ID NO:5) target polynucleotides to the liver with efficiency comparable to or superior to AAV8 and superior to Rh74 AAV in mice (see, e.g., FIG. 5). In addition, data in non-human primates show that AAV-Rh74 and AAV-Rh74 variants such as RHM4-1 are approximately two-fold more potent than AAV8 at mediating liver-derived expression of hFIX (see, e.g., FIGS. 4 and 6).

Thus, AAV-Rh74 and AAV-Rh74 variants such as capsid variants (e.g., RHM4-1) can be used to deliver polynucleotides, such as gene coding sequences, to express proteins that provide a desirable or therapeutic benefit, as well as for inhibitory nucleotides that reduce or inhibit expression of an undesirable or defective gene, thereby treating a variety of diseases. For example, AAV-Rh74 and AAV-Rh74capsid variants (e.g., RHM4-1) can be used as vectors to deliver to cells, tissues and organs, such as liver therapeutic genes (e.g., FIX, FVIII) to treat hemophilia A, B, etc. Such AAV-Rh74 and AAV-Rh74capsid variant (e.g., RHM4-1) vectors can also be used to deliver genes for a wide range of other metabolic or plasma protein deficiencies, or for other therapeutic purposes, such as but not limited to genes encoding zinc finger nucleases to carry out genome editing in the liver, and for local (liver) delivery of immunomodulatory agents such as alpha-interferon for treatment of hepatitis virus infections, or to treat virtually any disease that requires either liver transduction or presence of the therapeutic transgene product in the bloodstream (which can be achieved by targeting the transgenes for liver expression).

In addition to efficient delivery of polynucleotides by AAV-Rh74 and related AAV vectors such as AAV-Rh74 variants such as capsid variants (e.g., RHM4-1) into cells in vitro, ex vivo and in vivo, prevalence of anti-AAV-Rh74 antibodies in humans is lower than anti-AAV2 antibodies, and differs from that of anti-AAV8 antibodies (FIG. 7). Owing to low sero-prevalence, AAV-Rh74and related AAV vectors such as AAV-Rh74 (capsid) variants (e.g., RHM4-1) can be used in a greater percentage of humans which otherwise would not be eligible for gene transfer, for example, humans that may be sero-positive for other AAV serotypes (e.g., AAV1, AAV2, AAV3, AAV4, AAV5, AAV6, AAV7, AAV8, AAV9, AAV10, AAV11, etc.). In addition, AAV-Rh74 and related vectors such as AAV-Rh74 variants including capsid variants (e.g., RHM4-1) can be efficiently produced at high titers (Table 1). Thus, AAV-Rh74 and related AAV vectors can be produced in large amounts for more prevalent clinical diseases. In accordance with the invention, there are provided recombinant AAV-Rh74 vectors and related AAV vectors such as AAV-Rh74 variants such as capsid variants (e.g., RHM4-1) particles that include (encapsidate, package) AAV vector genomes. In one embodiment, a recombinant AAV vector includes a heterologous polynucleotide sequence. In another embodiment, a recombinant AAV vector genome is encapsidated or packaged by an AAV-Rh74 capsid or a related AAV such as AAV-Rh74 variants such as a capsid variant (e.g., RHM4-1).

In invention recombinant AAV vectors, such as AAV-Rh74 vectors and related AAV vectors such as AAV-Rh74 (capsid) variants (e.g., RHM4-1) particles that include (encapsidate, package) recombinant AAV vector genomes, the heterologous polynucleotide sequence may or be transcribed and subsequently translated into a protein. Alternatively, the heterologous polynucleotide may be transcribed into a transcript that in itself has a function or activity (e.g., as an inhibitory nucleic acid).

In various aspects, the heterologous polynucleotide sequence encodes a therapeutic protein. In particular aspects, the protein is a blood clotting factor (e.g., Factor XIII, Factor IX, Factor X, Factor VIII, Factor VIIa, or protein C), CFTR (cystic fibrosis transmembrane regulator protein), an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor (e.g., insulin-like growth factors 1 and 2, platelet derived growth factor, epidermal growth factor, nerve growth factor, neurotrophic factor -3 and -4, brain-derived neurotrophic factor, glial derived growth factor, transforming growth factor a and B, etc.), a cytokine (e.g., α-interferon, β-interferon, interferon-γ, interleukin-2, interleukin-4, interleukin 12, granulocyte-macrophage colony stimulating factor, lymphotoxin, etc.), a suicide gene product (e.g., herpes simplex virus thymidine kinase, cytosine deaminase, diphtheria toxin, cytochrome P450, deoxycytidine kinase, tumor necrosis factor, etc.), a drug resistance protein (e.g, that provides resistance to a drug used in cancer therapy), a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitopes, or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USHIC (Usher's Syndrome 1C), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, gene deficiencies causative of lysosomal storage diseases (e.g., sulfatases, N-acetylglucosamine-1-phosphate transferase, cathepsin A, GM2-AP, NPC1, VPC2, Sphingolipid activator proteins, etc.), one or more zinc finger nucleases for genome editing, or donor sequences used as repair templates for genome editing.

In additional aspects, the heterologous polynucleotide sequence encodes a therapeutic protein that in turn inhibits expression or function of an undesirable or aberrant (dysfunctional) protein present (endogenous) in a subject. In further aspects, the heterologous polynucleotide sequence is a polynucleotide which, when transcribed, is transcribed into an inhibitory nucleic acid (e.g., inhibitory RNA). In more particular aspects, an inhibitory nucleic acid is a single-stranded sequence, or forms a double-or triple-stranded sequence. In additional more particular aspects, an inhibitory nucleic acid is a micro-RNA (miRNA), siRNA, shRNA, trans-splicing RNA, antisense RNA or triplex forming RNA.

In further more particular aspects, an inhibitory nucleic acid inhibits expression of: huntingtin (HTT) gene, a gene associated with dentatorubropallidolusyan atropy (e.g., atrophin 1, ATN1); androgen receptor on the X chromosome in spinobulbar muscular atrophy, human Ataxin-1, -2, -3, and -7, Ca.2.1 P/Q voltage-dependent calcium channel is encoded by the (CACNAIA), TATA-binding protein, Ataxin 8 opposite strand, also known as ATXN8OS, Serine/threonine-protein phosphatase 2A 55 kDa regulatory subunit B beta isoform in spinocerebellar ataxia (type 1, 2, 3, 6, 7, 8, 12 17), FMRI (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercoloesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7,also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DAN damage-inducible transcript 4 protein, in diabetic macular oedma (DME) or age-related macular degeneration; vascular endothelial growth factor receptor I (VEGFR1) in age-related macular degeneration or choroidal neivascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TORIA) in primary dystonia, pan-class I and HLA-allele specific in transplant; mutant rhodopsin gene (RHO) in autosomal dominantly inherited retinitis pigmentosa (adRP); or the inhibitory nucleic acid binds to a transcript of any of the foregoing genes or sequences.

Invention recombinant AAV vectors and AAV-Rh74 and related AAV vectors such as AAV-Rh74 vector variants such as capsid variants (e.g., RHM4-1) particles that include (encapsidate, package) recombinant AAV vector genome include additional elements that function in cis or in trans. In particular embodiments, a recombinant viral (e.g., AAV) vector and/or AAV-Rh74 vector or related AAV vector such as a AAV-Rh74 (capsid) variants (e.g., RHM4-1) particle that includes (encapsidate, package) recombinant AAV vector genome also has: one or more inverted terminal repeat (ITR) sequences that flank the 5′ or 3′ terminus of the heterologous polynucleotide sequence; an expression control sequence that drives transcription of the heterologous polynucleotide sequence (e.g., a promoter or enhancer that contributes to transcription of the heterologous polynucleotide sequence, such as a constitutive or regulatable control element, or tissue-specific expression control element); a poly-Adenine sequence located 3′ of the heterologous polynucleotide sequence; a selectable marker (e.g., a protein that provides antibiotic resistance, such as Kanamycin resistance); and/or an origin of replication.

Invention recombinant AAV vectors and AAV-Rh74 vectors and related AAV vectors such as AAV-Rh74 variants including capsid variant (e.g., RHM4-1) particles that include (encapsidate, package) recombinant AAV vector genome can also include additional elements. In one embodiment, a recombinant vector genome includes a heterologous polynucleotide sequence and a filler or stuffer polynucleotide sequence. In particular aspects, a heterologous polynucleotide sequence has a length less than about 4.7 Kb. In further particular aspects, a heterologous polynucleotide sequence has a length less than 4.7 Kb and is positioned within two adeno-associated virus (AAV) ITR sequences. In additional particular aspects, a filler or stuffer polynucleotide sequence has a length that when combined with the heterologous polynucleotide sequence the total combined length of the heterologous polynucleotide sequence and filler or stuffer polynucleotide sequence is between about 3.0-5.5 Kb, or between about 4.0-5.0 Kb, or between about 4.3-4.8 Kb.

Filler or stuffer polynucleotide sequences can be located in the vector sequence at any desired position such that it does not prevent a function or activity of the vector. In one aspect, a filler or stuffer polynucleotide sequence is not positioned between a 5′ and/or 3′ ITR that flanks the respective 5′ and/or 3′ termini of a heterologous polynucleotide sequence. In another aspect, a filler or stuffer polynucleotide sequence is positioned within a 5′ and/or 3′ ITR that flanks the respective 5′ and/or 3′ termini of a heterologous polynucleotide sequence. In an additional aspect, a filler or stuffer polynucleotide sequence is positioned adjacent to 5′ and/or 3′ ITR that flanks the respective 5′ and/or 3′ termini of a heterologous polynucleotide sequence. In a further aspect, a filler or stuffer polynucleotide sequence is positioned within a heterologous polynucleotide sequence, e.g., analogous to an intron within a genomic nucleic acid.

Accordingly, in various embodiments, a filler or stuffer polynucleotide sequence is positioned adjacent to an AAV ITR sequence; positioned within two adeno-associated virus (AAV) ITR sequences; positioned outside two adeno-associated virus (AAV) ITR sequences; or there are two filler or stuffer polynucleotide sequences, a first filler or stuffer polynucleotide sequence positioned within two adeno-associated virus (AAV) ITR sequences, and a second filler or stuffer polynucleotide sequence positioned outside two adeno-associated virus (AAV) ITR sequences.

In more particular aspects, a filler or stuffer polynucleotide sequence has a length that when combined with said heterologous polynucleotide sequence the total combined length of the heterologous polynucleotide sequence and filler or stuffer polynucleotide sequence is between about 3.0-5.5 Kb, between about 4.0-5.0 Kb, or between about 4.3-4.8 Kb, when positioned within two adeno-associated virus (AAV) ITR sequences. In other more particular aspects, a filler or stuffer polynucleotide sequence has a length greater than 4.7 Kb, between about 5.0-10.0 Kb, or between about 6.0-8.0 Kb, when positioned outside two adeno-associated virus (AAV) ITR sequences.

In various additional aspects, a filler or stuffer polynucleotide sequence is a sequence between about 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000 nucleotides in length.

Typically, a filler or stuffer polynucleotide sequence is inert or innocuous and has no function or activity. In various particular aspects, a filler or stuffer polynucleotide sequence is not a bacterial polynucleotide sequence, a filler or stuffer polynucleotide sequence is not a sequence that encodes a protein or peptide, a filler or stuffer polynucleotide sequence is a sequence distinct from any of: the heterologous polynucleotide sequence, an AAV inverted terminal repeat (ITR) sequence, an expression control element, an origin of replication, a selectable marker or a poly-Adenine (poly-A) sequence.

In various additional particular aspects, a filler or stuffer polynucleotide sequence is an intron sequence that is related to or unrelated to the heterologous polynucleotide sequence. In particular aspects, the intron sequence is positioned within the heterologous polynucleotide sequence. In other particular aspects, the intron sequence is related to the heterologous polynucleotide sequence as the intron is in genomic DNA, such as the genomic DNA that encodes a protein which protein is also encoded by the heterologous polynucleotide sequence.

Invention recombinant AAV vectors and AAV-Rh74 and related AAV vectors such as AAV-Rh74 variants such as capsid variant (e.g., RHM4-1) particles that include (encapsidate, package) recombinant AAV vector genome can be included within cells. In such embodiments, cells can comprise helper cells lysed to produce virus (AAV) particles (e.g., AAV-Rh74 vectors or related AAV vectors such as AAV-Rh74 capsid variants (e.g., RHM4-1)), or target cells in which it is desired to express the heterologous polynucleotide sequence.

Invention recombinant AAV vectors and AAV-Rh74 vectors and related AAV vectors such as AAV-Rh74 variants (e.g., capsid variants such as RHM4-1) particles that include (encapsidate, package) recombinant AAV vector genome can be included within pharmaceutical compositions. Such compositions are useful for administration of recombinant vector (e.g., AAV) and virus particles such as AAV-Rh74 vectors and related AAV vectors such as AAV-Rh74 variants (e.g., capsid variants such as RHM4-1) that include (encapsidate, package) recombinant vector (e.g., AAV) genomes to a subject.

Invention recombinant AAV vectors and AAV-Rh74 vectors and related AAV vectors such as AAV-Rh74 variants (e.g., capsid variants such as RHM4-1) particles that include (encapsidate, package) recombinant AAV vector genome may be employed in various methods and uses. Accordingly, there are provided methods and uses for delivering or transferring a heterologous polynucleotide sequence into an organism or cell, such as a mammal or a cell of a mammal.

In one embodiment, a method or use includes administering an adeno-associated virus (AAV) vector that includes a heterologous polynucleotide sequence (e.g., via an AAV-Rh74 vector or related AAV such as AAV-Rh74 variants (e.g., capsid variants such as RHM4-1)) particle that includes (encapsidate, package) the vector genome to a mammal or a cell of a mammal under suitable conditions to deliver or transfer the heterologous polynucleotide sequence into the mammal or the cell of a mammal. In one aspect, the method or use allows transfer/delivery of the heterologous polynucleotide into the mammal and/or cell. In another aspect, the method allows transfer/delivery of the heterologous polynucleotide into the mammal and/or cell, and subsequent transcription of the heterologous polynucleotide thereby forming a transcript. In a further aspect, the method allows transfer/delivery of the heterologous polynucleotide into the cell, subsequent transcription to form a transcript and subsequent translation to form a gene product (protein). In particular, for example, in the latter two aspects a heterologous polynucleotide sequence is operably linked to an expression control element conferring transcription of the heterologous polynucleotide sequence, and optionally subsequent translation of the transcript.

In additional embodiments, a method or use is for delivering or transferring a heterologous polynucleotide sequence into a subject (e.g., mammal) or a cell of a subject (e.g., mammal), and includes administering a viral (e.g., AAV-Rh74 or related AAV such as AAV-Rh74 capsid variants (e.g., RHM4-1)) particle, a plurality of such viral (e.g., AAV) particles, or a pharmaceutical composition of such a viral (e.g., AAV-Rh74 or related AAV such as AAV-Rh74 capsid variants (e.g., RHM4-1)) particle or plurality of such viral (e.g., AAV) particles to a subject (e.g., mammal) or a cell of the subject (e.g., mammal), thereby delivering or transferring a heterologous polynucleotide sequence into the subject (e.g., mammal) or cell of the subject (e.g., mammal). In another embodiment, a method or use is for treating a subject (e.g., mammal) deficient or in need of protein expression or function, or in need of reduced expression or function of an endogenous protein (e.g., an undesirable, aberrant or dysfunctional protein), that includes providing a viral (e.g., AAV-Rh74 and related AAV such as AAV-Rh74 variants (e.g., capsid variants such as RHM4-1)) particle, a plurality of such viral (e.g., AAV) particles, or a pharmaceutical composition of a viral (e.g., AAV-Rh74 and related AAV such as AAV-Rh74 variants (e.g., capsid variants such as RHM4-1)) particle or plurality of such viral (e.g., AAV) particles; and administering the viral (e.g., AAV-Rh74 and related AAV such as AAV-Rh74 variants (e.g., capsid variants such as RHM4-1)) particle, plurality of such viral (e.g., AAV) particles, or pharmaceutical composition of viral (e.g., AAV-Rh74 and related AAV such as AAV-Rh74 variants (e.g., capsid variants such as RHM4-1)) particle or plurality of such viral (e.g., AAV) particles to the subject (e.g., mammal), where the heterologous polynucleotide sequence is expressed in the mammal, or wherein the heterologous polynucleotide sequence encodes an inhibitory sequence or protein that reduces expression or function of an endogenous protein (e.g., an undesirable, aberrant or dysfunctional protein) in the subject (e.g., mammal).

Methods and uses for administration or delivery include any mode compatible with a subject. In particular embodiments, a viral (e.g., AAV-Rh74 or related AAV such as AAV-Rh74 capsid variant (e.g., RHM4-1)) particle or plurality of such viral (e.g., AAV) particles (e.g., AAV-Rh74 vectors or related AAV vectors such as AAV-Rh74 capsid variants (e.g., RHM4-1)) is administered or delivered intravenously, intraarterially, intramuscularly, subcutaneously, orally, by intubation, via catheter, dermally, intra-cranially, via inhalation, intra-cavity, or mucosally.

Subjects include mammals, such as humans and non-humans (e.g., primates). In particular embodiments, a subject would benefit from or is in need of expression of a heterologous polynucleotide sequence.

In accordance with the invention, methods of producing recombinant vector (e.g., AAV) plasmids and virus (e.g., AAV-Rh74 and related AAV such as AAV-Rh74 variants (e.g., capsid variants such as RHM4-1)) particles that include (encapsidate, package) recombinant vector (e.g., AAV) are provided. In one embodiment, a method of producing recombinant viral or AAV particles includes introducing into a packaging helper cell a recombinant vector (e.g., AAV) plasmid to produce a productive viral (e.g., AAV-Rh74 and related AAV such as AAV-Rh74 variants (e.g., capsid variants such as RHM4-1)) infection; and culturing the helper cells under conditions to produce recombinant viral (e.g., AAV-Rh74 or related AAV such as AAV-Rh74 capsid variants (e.g., RHM4-1)) particles. In another embodiment, a method of producing recombinant viral or AAV particles with reduced amounts of recombinant viral (e.g., AAV-Rh74 and related AAV such as AAV-Rh74 variants (e.g., capsid variants such as RHM4-1)) particles in which the recombinant viral vector includes contaminating nucleic acid, includes introducing into a packaging helper cell a recombinant vector (e.g., AAV) plasmid; and culturing the helper cells under conditions to produce recombinant viral (e.g., AAV-Rh74 and related AAV such as AAV-Rh74 variants (e.g., capsid variants such as RHM4-1)) particles, wherein the recombinant viral (e.g., AAV-Rh74 and related AAV such as AAV-Rh74 variants (e.g., capsid variants such as RHM4-1)) particles produced have reduced numbers of viral (e.g., AAV-Rh74 and related AAV such as AAV-Rh74 variants (e.g., capsid variants such as RHM4-1)) particles with recombinant vector genomes that contain contaminating nucleic acid compared to the numbers of viral (e.g., AAV-Rh74 and related AAV such as AAV-Rh74 variants (e.g., capsid variants such as RHM4-1)) particles that contain contaminating nucleic acid produced under conditions in which the filler or stuffer polynucleotide sequence is absent from the recombinant viral vector. In particular aspects, the contaminating nucleic acid is bacterial nucleic acid; or a sequences other than the heterologous polynucleotide sequence, or ITR, promoter, enhancer, origin of replication, poly-Adenine sequence, or selectable marker.

Helper cells include mammalian cells. In particular embodiments, a helper cell provides helper (e.g., AAV) functions to package the heterologous polynucleotide sequence into a viral particle (e.g., AAV particle such as AAV-Rh74 and related AAV vectors such as AAV-Rh74 variants (e.g., capsid variants such as RHM4-1)). In particular aspects, a helper cell provides AAV Rep and/or Cap proteins (e.g., Rep78 or/and Rep68 proteins); a helper cell is stably or transiently transfected with polynucleotide(s) encoding Rep and/or Cap protein sequence(s); a helper cell is stably or transiently transfected with Rep78 and/or Rep68 protein polynucleotide encoding sequence(s).

Invention recombinant vector (e.g., AAV) plasmids can be based upon any strain or serotype, including hybrids or chimeras of different serotypes. Invention recombinant viral (e.g., AAV) particles are typically based upon AAV-Rh74 or related AAV such as AAV-Rh74 and related AAV such as AAV-Rh74 variants (e.g., capsid variants such as RHM4-1)), but also include hybrids or chimeras with different serotypes. Representative AAV serotypes include, without limitation, AAV1, AAV2, AAV3, AAV4, AAV5, AAV6, AAV7, AAV8, AAV9, AAV10, AAV11, and Rh10 serotypes. Accordingly, invention recombinant viral (e.g., AAV) particles comprising vector genomes can include a capsid protein from a different serotype, a mixture of serotypes, or hybrids or chimeras of different serotypes, such as a VP1, VP2 or VP3 capsid protein of AAV1, AAV2, AAV3, AAV4, AAV5, AAV6, AAV7, AAV8, AAV9, AAV10, AAV11, Rh10 serotype. Furthermore, invention recombinant vectors (e.g., AAV), sequences, plasmids, vector genomes, can include elements from any one serotype, a mixture of serotypes, or hybrids or chimeras of different serotypes. In various embodiments, a recombinant AAV vector includes a Cap, Rep, and/or ITR sequence derived from AAV1, AAV2, AAV3, AAV4, AAV5, AAV6, AAV7, AAV8, AAV9,AAV10, AAV11, Rh74, or Rh10 serotype, or a mixture, hybrid or chimera of any of the foregoing AAV serotypes.

The patent or application file contains at least one drawing executed in color. Copies of this patent or patent application publication with color drawing(s) will be provided by the Office upon request and payment of the necessary fee.

### DESCRIPTION OF DRAWINGS

FIG. 1 shows human factor IX (FIX) plasma levels in C57BL/6 mice (n=5 per group) injected via the tail vein with AAV vectors expressing the FIX transgene under the control of a liver-specific promoter. Vector dose 2.5¹⁰ vector genomes per mouse. FIX transgene product (FIX protein) plasma levels were measured by ELISA at weeks 1, 2, and 4 post gene transfer. AAV-Rh74 conferred the highest levels of FIX transgene expression.

FIG. 2 shows canine FIX plasma levels in hemophilia B dogs after the delivery of 3¹² vector genomes per kilogram (kg) of weight. AAV vectors were infused intravenously (IV) though the saphenous vein and FIX levels were monitored by ELISA. Expression of the therapeutic FIX transgene was driven by a liver specific promoter. AAV8 and AAV-Rh74 vectors performed roughly equally in hemophilia B dogs and were both superior to AAV6.

FIGS. 3A-3B show AAV-Rh74 VP1, VP2, and VP3 amino acid sequences and, for VP1, polynucleotide (DNA) sequence (SEQ ID NOs: 1-4).

FIG. 4 shows administration of AAV8 and AAVrh74 vector expressing human Factor IX (FIX) (under the control of a liver-specific promoter) to rhesus macaques, a non-human primate, and expression of FIX in the animals. Animals receiving the AAVrh74-FIX vectors (last two bars towards the right margin) expressed the FIX transgene at higher levels compared to the other groups of animals injected at the same dose.

FIG. 5 shows plasma human factor IX expression levels in animals administered AAV human factor IX expression vector encapsidated by the indicated AAV (Rh74 variants, such as RHM4-1 variant), compared to Rh74 and AAV8 encapsidated AAV human factor IX expression vector.

FIG. 6 shows administration of AAV8 and AAV-Rh74 variant RHM4-1 vector expressing human Factor IX (under the control of a liver-specific promoter) to cynomolgus macaques, a non-human primate, and expression of FIX in the animals.

FIG. 7A-7B show anti-AAV neutralizing antibodies (NAb) measured in humans with an in vitro assay.

### DETAILED DESCRIPTION

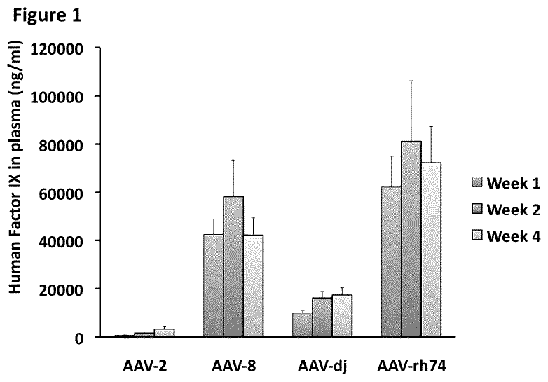

The invention is based, at least in part, on data indicating that adeno-associated virus (AAV) serotype AAV-Rh74 and related AAV variants such as AAV-Rh74 capsid variants (e.g., RHM4-1, RHM15-1, RHM15-2, RHM15-3/RHM15-5, RHM15-4 and RHM15-6) have a high tropism for hepatocytes, which are cells of the liver. As a vector for polynucleotide (e.g. genes, inhibitory nucleic acid, etc.) transfer/delivery into cells, AAV-Rh74 and related AAV variants such as AAV-Rh74 variants (e.g., capsid variants such as RHM4-1, RHM15-1, RHM15-2, RHM15-3/RHM15-5, RHM15-4 and RHM15-6) can be used to provide therapeutic levels of expression in liver after intravenous administration. Furthermore, AAV-Rh74 vectors and related AAV variants such as AAV-Rh74 variants (e.g., capsid variants such as RHM4-1, RHM15-1, RHM15-2, RHM15-3/RHM15-5, RHM15-4 and RHM15-6) mediated gene transfer/delivery produced protein expression levels that were significantly higher than several other serotypes (see, e.g., FIGS. 1, 2, 4, 5 and 6). In particular, AAV-Rh74 vector and related AAV-Rh74 capsid variants (e.g., RHM4-1) target genes for delivery to the liver with efficiency at least comparable and typically superior to the gold standard for liver transduction, AAV8, in hemophilia B dogs and/or in mice and/or macaques. Thus, AAV-Rh74 vectors and related AAV variants such as AAV-Rh74 capsid variants (e.g., RHM4-1) can be used to transfer/deliver heterologous polynucleotides, such as coding sequences (genes) for proteins that provide a desirable or therapeutic benefit, as well as inhibitory (e.g., anti-sense) nucleic acid that reduce or inhibit expression of an undesirable or defective (e.g., pathologic) gene, thereby treating a variety of diseases. For example, a recombinant vector (e.g., AAV) genome can be packaged or encapsidated within AAV-Rh74 vector or related AAV variants such as AAV-Rh74 capsid variants (e.g., RHM4-1, RHM15-1, RHM15-2, RHM15-3/RHM15-5, RHM15-4 and RHM15-6) in order to transfer/deliver a heterologous polynucleotide into a cell.

As set forth herein, adeno-associated virus (AAV) serotype AAV-Rh74 and related AAV variants such as AAV-Rh74 capsid variants (e.g., RHM4-1, RHM15-1, RHM15-2, RHM15-3/RHM15-5, RHM15-4 and RHM15-6) provide a means for delivery of polynucleotide sequences into cells ex vivo, in vitro and in vivo. Such polynucleotide sequences can encode proteins such that the cells into which the polynucleotides are delivered express the encoded proteins. For example, a recombinant AAV vector can include heterologous polynucleotides encoding a desired protein or peptide, or a heterologous polynucleotide that when transcribed comprises an inhibitory sequence (e.g., RNA), for example, a sequence that targets a gene for inhibition of expression. Vector delivery or administration to a subject (e.g., mammal) therefore provides not only heterologous polynucleotides encoding proteins and peptides to the subject, but also inhibitory nucleic acids that target genes for inhibition of expression or function in the subject.

Thus, in accordance with the invention AAV-Rh74 vectors and related AAV vector variants such as AAV-Rh74 variants (e.g., capsid variants such as RHM4-1, RHM15-1, RHM15-2, RHM15-3/RHM15-5, RHM15-4 and RHM15-6) that include (encapsidate or package) vector genome including polynucleotide sequences encoding peptides and proteins, as well as polynucleotide sequences which directly or when transcribed comprise inhibitory nucleic acids that target genes for inhibition of expression or function, are provided.

A recombinant “vector” or “AAV vector” is derived from the wild type genome of a virus, such as AAV by using molecular methods to remove the wild type genome from the virus (e.g., AAV), and replacing with a non-native nucleic acid, such as a heterologous polynucleotide sequence (e.g., a therapeutic gene expression cassette). Typically, for AAV one or both inverted terminal repeat (ITR) sequences of the wild type AAV genome are retained in the AAV vector. A recombinant viral vector (e.g., AAV) is distinguished from a viral (e.g., AAV) genome, since all or a part of the viral genome has been replaced with a non-native sequence with respect to the viral (e.g., AAV) genomic nucleic acid such as a heterologous polynucleotide sequence. Incorporation of a non-native sequence such as a heterologous polynucleotide therefore defines the viral vector (e.g., AAV) as a “recombinant” vector, which in the case of AAV can be referred to as an “rAAV vector.”

A recombinant vector (e.g., AAV) sequence can be packaged into a virus (also referred to herein as a “particle” or “virion”) for subsequent infection (transformation) of a cell, ex vivo, in vitro or in vivo. Where a recombinant vector sequence is encapsidated or packaged into an AAV particle, the particle can be referred to as a “rAAV.” Such particles or virions will typically include proteins that encapsidate or package the vector genome. Particular examples include viral envelope proteins, and in the case of AAV capsid proteins.

In particular embodiments, a recombinant vector (e.g., AAV) is a parvovirus vector. Parvoviruses are small viruses with a single-stranded DNA genome. “Adeno-associated viruses” (AAV) are in the parvovirus family.

Parvoviruses including AAV are viruses useful as gene therapy vectors as they can penetrate cells and introduce nucleic acid/genetic material so that the nucleic acid/genetic material may be stably maintained in cells. In addition, these viruses can introduce nucleic acid/genetic material into specific sites, for example, such as a specific site on chromosome 19. Because AAV are not associated with pathogenic disease in humans, AAV vectors are able to deliver heterologous polynucleotide sequences (e.g., therapeutic proteins and agents) to human patients without causing substantial AAV pathogenesis or disease.

AAV-Rh74 and related AAV variants such as AAV-Rh74 or related AAV such as AAV-Rh74 variants (e.g., capsid variants such as RHM4-1) serotypes (e.g., VP1, VP2, and/or VP3sequences) may or may not be distinct from other AAV serotypes, including, for example, AAV1-AAV11 or Rh10 (e.g., distinct from VP1, VP2, and/or VP3 sequences of any of AAV1-AAV11, or Rh10 serotypes). As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV-Rh74 variants including capsid variants may not be serologically distinct from Rh74 or other AAV, they differ by at least one nucleotide or amino acid residue compared to Rh74 or other AAV.

Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

Recombinant vector (e.g., AAV) plasmids, as well as methods and uses thereof, include any viral strain or serotype. As a non-limiting example, a recombinant vector (e.g., AAV) plasmid can be based upon any AAV genome, such as AAV-1, -2, -3, -4, -5, -6, -7, -8, -9, -10, -11, -rh74, -rh10 or AAV-218, for example. Such vectors can be based on the same of strain or serotype (or subgroup or variant), or be different from each other. As a non-limiting example, a recombinant vector (e.g., AAV) plasmid based upon one serotype genome can be identical to one or more of the capsid proteins that package the vector. In addition, a recombinant vector (e.g., AAV) plasmid can be based upon an AAV (e.g., AAV2) serotype genome distinct from one or more of the capsid proteins that package the vector, in which case at least one of the three capsid proteins could be a AAV-Rh74 or related AAV variant such as AAV-Rh74 or related AAV such as AAV-Rh74 variants (e.g., capsid variants such as RHM4-1, RHM15-1, RHM15-2, RHM15-3/RHM15-5, RHM15-4 and RHM15-6), for example.

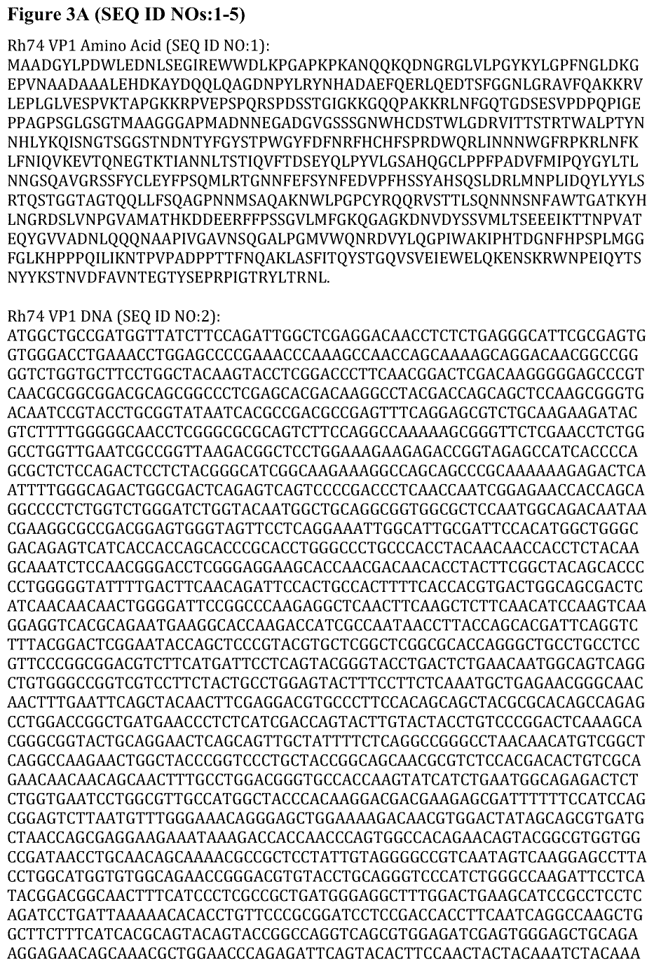

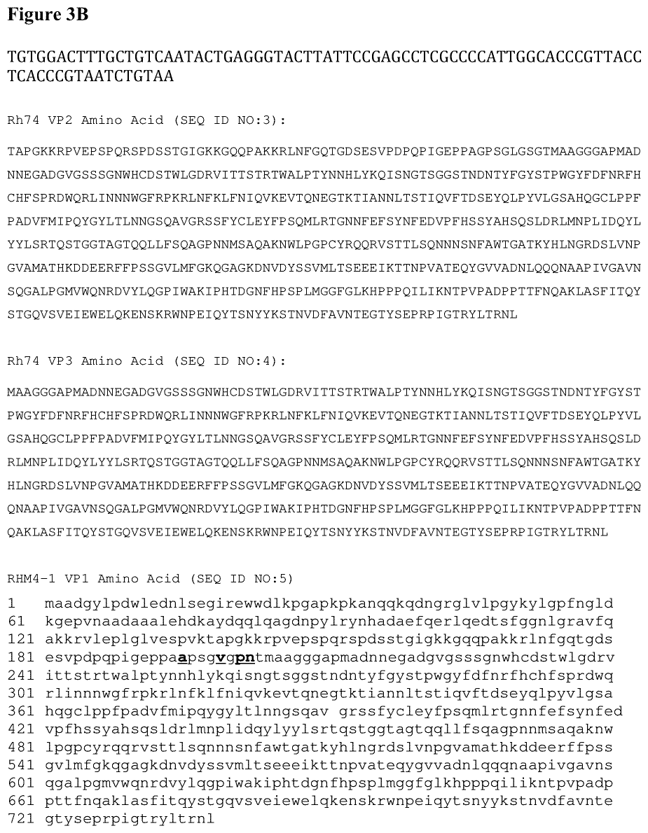

AAV-Rh74 has gene/protein sequences identical to sequences characteristic for AAV-Rh74 (see, e.g., VP1, VP2, VP3 of FIG. 3). As used herein, an “AAV vector related to AAV-Rh74” and grammatical variations thereof refers to one or more AAV proteins (e.g., VP1, VP2, and/or VP3 sequences) that has substantial sequence identity to one or more polynucleotides or polypeptide sequences that comprise AAV-Rh74. Such AAV vectors related to AAV-Rh74 can therefore have one or more distinct sequences from AAV-Rh74, but can exhibit substantial sequence identity to one or more genes and/or have one or more functional characteristics of AAV-Rh74 (e.g., such as cell/tissue tropism). For example, AAV-Rh74 related AAV variant RHM4-1 has a capsid with four amino acids different from Rh74 capsid. Exemplary AAV-Rh74 and related AAV variants such as AAV-Rh74 or related AAV such as AAV-Rh74 variants (e.g., capsid variants such as RHM4-1, RHM15-1, RHM15-2, RHM15-3/RHM15-5, RHM15-4 and RHM15-6) sequences include VP1, VP2, and/or VP3 set forth herein, for example, in FIG. 3. In one non-limiting exemplary embodiment, an AAV vector related to AAV-Rh74 has a polynucleotide, polypeptide or subsequence thereof that includes or consists of a sequence at least 80% or more (e.g., 85%, 90%, 95%, 96%, 97%, 98%, 99%, 99.5%, etc.) identical to one or more AAV-Rh74 VP1, VP2, and/or VP3 sequences set forth in FIG. 3.

In accordance with the invention, methods and uses include AAV-Rh74 sequences (polypeptides and nucleotides) and subsequences thereof that exhibit less than 100% sequence identity to a reference AAV-Rh74 gene or protein sequence (e.g., VP1, VP2, and/or VP3 sequences set forth in FIG. 3), but are distinct from and not identical to known AAV genes or proteins, such as AAV1-AAV11, AAV-Rh10, genes or proteins, etc. In one embodiment, an AAV-Rh74 polypeptide or subsequence thereof includes or consists of a sequence at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical to any reference AAV-Rh74 sequence or subsequence thereof (e.g., VP1, VP2 and/or VP3 sequences set forth in FIG. 3). In particular aspects, an AAV-Rh74 related variant has one, two, three or four of the four amino acid substitutions set forth in AAV-Rh74 (e.g., capsid variant RHM4-1, RHM15-1, RHM15-2, RHM15-3/RHM15-5, RHM15-4 and RHM15-6).

Recombinant vectors (e.g., AAV), including AAV1, AAV2, AAV3, AAV4, AAV5, AAV6, AAV7, AAV8, AAV9, AAV10, AAV11, Rh10, Rh74 or AAV-218 and variant, related, hybrid and chimeric sequences, can be constructed using recombinant techniques that are known to the skilled artisan, to include one or more heterologous polynucleotide sequences (transgenes) flanked with one or more functional AAV ITR sequences. Such vectors can have one or more of the wild type AAV genes deleted in whole or in part, for example, a rep and/or cap gene, but retain at least one functional flanking ITR sequence, as necessary for the rescue, replication, and packaging of the recombinant vector into an AAV vector particle. An AAV vector genome would therefore include sequences required in cis for replication and packaging (e.g., functional ITR sequences)

The terms “polynucleotide” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, IRNA tRNA and inhibitory DNA or RNA (RNAi, e.g., small or short hairpin (sh) RNA, microRNA (miRNA), small or short interfering (si) RNA, trans-splicing RNA, or antisense RNA). Polynucleotides include naturally occurring, synthetic, and intentionally altered or modified polynucleotides as well as analogues and derivatives. Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

A “heterologous” polynucleotide refers to a polynucleotide inserted into a vector (e.g., AAV) for purposes of vector mediated transfer/delivery of the polynucleotide into a cell. Heterologous polynucleotides are typically distinct from vector (e.g., AAV) nucleic acid, i.e., are non-native with respect to viral (e.g., AAV) nucleic acid. Once transferred/delivered into the cell, a heterologous polynucleotide, contained within the virion, can be expressed (e.g., transcribed, and translated if appropriate). Alternatively, a transferred/delivered heterologous polynucleotide in a cell, contained within the virion, need not be expressed. Although the term “heterologous” is not always used herein in reference to polynucleotides, reference to a polynucleotide even in the absence of the modifier “heterologous” is intended to include heterologous polynucleotides in spite of the omission.

The “polypeptides,” “proteins” and “peptides” encoded by the “polynucleotide sequences,” include full-length native sequences, as with naturally occurring proteins, as well as functional subsequences, modified forms or sequence variants so long as the subsequence, modified form or variant retains some degree of functionality of the native full-length protein. In methods and uses of the invention, such polypeptides, proteins and peptides encoded by the polynucleotide sequences can be but are not required to be identical to the endogenous protein that is defective, or whose expression is insufficient, or deficient in the treated mammal.

Invention adeno-associated virus (AAV) serotype AAV-Rh74 and related AAV variants such as AAV-Rh74 variants (e.g., capsid variants such as RHM4-1, RHM4-1, RHM15-1, RHM15-2, RHM15-3/RHM15-5, RHM15-4 and RHM15-6), can be used to introduce/deliver polynucleotides stably or transiently into cells and progeny thereof. The term “transgene” is used herein to conveniently refer to such a heterologous polynucleotide that has been introduced into a cell or organism. Transgenes include any polynucleotide, such as a gene that encodes a polypeptide or protein, a polynucleotide that is transcribed into an inhibitory polynucleotide, or a polynucleotide that is not transcribed (e.g., lacks a expression control element, such as a promoter that drives transcription).

For example, in a cell having a transgene, the transgene has been introduced/transferred by way of vector, such as AAV, “transformation” of the cell. The terms “transform,” and “transfect” refer to introduction of a molecule such as a polynucleotide into a cell or host organism.

A cell into which the transgene has been introduced is referred to as a “transformed cell” or “transformant.” Accordingly, a “transformed” or “transfected” cell (e.g., in a mammal, such as a cell or tissue or organ cell), means a genetic change in a cell following incorporation of an exogenous molecule, for example, a polynucleotide or protein (e.g., a transgene) into the cell. Thus, a “transfected” or “transformed” cell is a cell into which, or a progeny thereof in which an exogenous molecule has been introduced, for example. The cell(s) can be propagated and the introduced protein expressed, or nucleic acid transcribed. For gene therapy uses and methods, a transformed cell can be in a subject.

The introduced polynucleotide may or may not be integrated into nucleic acid of the recipient cell or organism. If an introduced polynucleotide becomes integrated into the nucleic acid (genomic DNA) of the recipient cell or organism it can be stably maintained in that cell or organism and further passed on to or inherited by progeny cells or organisms of the recipient cell or organism. Finally, the introduced nucleic acid may exist in the recipient cell or host organism only transiently.

Cells that may be transformed include a cell of any tissue or organ type, of any origin (e.g., mesoderm, ectoderm or endoderm). Non-limiting examples of cells include liver (e.g., hepatocytes, sinusoidal endothelial cells), pancreas (e.g., beta islet cells), lung, central or peripheral nervous system, such as brain (e.g., neural, glial or ependymal cells) or spine, kidney, eye (e.g., retinal, cell components), spleen, skin, thymus, testes, lung, diaphragm, heart (cardiac), muscle or psoas, or gut (e.g., endocrine), adipose tissue (white, brown or beige), muscle (e.g., fibroblasts), synoviocytes, chondrocytes, osteoclasts, epithelial cells, endothelial cells, salivary gland cells, inner ear nervous cells or hematopoietic (e.g., blood or lymph) cells. Additional examples include stem cells, such as pluripotent or multipotent progenitor cells that develop or differentiate into liver (e.g., hepatocytes, sinusoidal endothelial cells), pancreas (e.g., beta islet cells), lung, central or peripheral nervous system, such as brain (e.g., neural, glial or ependymal cells) or spine, kidney, eye (retinal, cell components), spleen, skin, thymus, testes, lung, diaphragm, heart (cardiac), muscle or psoas, or gut (e.g., endocrine), adipose tissue (white, brown or beige), muscle (e.g., fibroblasts), synoviocytes, chondrocytes, osteoclasts, epithelial cells, endothelial cells, salivary gland cells, inner ear nervous cells or hematopoietic (e.g., blood or lymph) cells.

A “therapeutic molecule” in one embodiment is a peptide or protein that may alleviate or reduce symptoms that result from an absence or defect in a protein in a cell or subject. Alternatively, a “therapeutic” peptide or protein encoded by a transgene is one that confers a benefit to a subject, e.g., to correct a genetic defect, to correct a gene (expression or functional) deficiency, or an anti-cancer effect.

Particular non-limiting examples of heterologous polynucleotides encoding gene products (e.g., therapeutic proteins) which are useful in accordance with the invention include, but are not limited to: genes that comprise or encode CFTR (cystic fibrosis transmembrane regulator protein), a blood coagulation (clotting) factor (Factor XIII, Factor IX, Factor X, Factor VIII, Factor VIIa, protein C etc.) including gain of function blood coagulation factors, an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor (e.g., insulin-like growth factors 1 and 2, platelet derived growth factor, epidermal growth factor, nerve growth factor, neurotrophic factor -3 and -4, brain-derived neurotrophic factor, glial derived growth factor, transforming growth factor α and β, etc.), a cytokine (e.g., α-interferon, β-interferon, interferon-γ, interleukin-2, interleukin-4, interleukin 12, granulocyte-macrophage colony stimulating factor, lymphotoxin, etc.), a suicide gene product (e.g., herpes simplex virus thymidine kinase, cytosine deaminase, diphtheria toxin, cytochrome P450, deoxycytidine kinase, tumor necrosis factor, etc.), a drug resistance protein (e.g, that provides resistance to a drug used in cancer therapy), a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitopes [de Groot et al., Blood 2008 Oct. 15;112(8):3303], or hCDR1 [Sharabi et al., Proc Natl Acad Sci U S A. 2006 Jun. 6;103(23):8810-5], insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USHIC (Usher's Syndrome 1C), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, gene deficiencies causative of lysosomal storage diseases (e.g., sulfatases, N-acetylglucosamine-1-phosphate transferase, cathepsin A, GM2-AP, NPC1, VPC2, Sphingolipid activator proteins, etc.), one or more zinc finger nucleases for genome editing, or donor sequences used as repair templates for genome editing.

Further non-limiting examples of heterologous polynucleotides encoding gene products (e.g., therapeutic proteins) which are useful in accordance with the invention include those that may be used in the treatment of a disease or disorder including, but not limited to, cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Gaucher's disease, Hurler's disease, adenosine deaminase deficiency, glycogen storage diseases and other metabolic defects, retinal degenerative diseases (and other diseases of the eye), and diseases of solid organs (e.g., brain, liver, kidney, heart).

All mammalian and non-mammalian forms of polynucleotides encoding gene products, including the non-limiting genes and proteins disclosed herein are expressly included, either known or unknown. Thus, the invention includes genes and proteins from non-mammals, mammals other than humans, and humans, which genes and proteins function in a substantially similar manner to the human genes and proteins described herein. A non-limiting example of non-mammalian gene is a Fok nuclease domain, which is bacterial in origin. Non-limiting examples of mammalian non-human FIX sequences are described in Yoshitake et al., 1985, supra; Kurachi et al., 1995, supra; Jallat et al., 1990, supra; Kurachi et al., 1982, Proc. Natl. Acad. Sci. USA 79:6461-6464; Jaye et al., 1983, Nucl. Acids Res. 11:2325-2335; Anson et al., 1984, EMBO J. 3:1053-1060; Wu et al., 1990, Gene 86:275-278; Evans et al., Proc Natl Acad Sci USA 86:10095 (1989), Blood 74:207-212; Pendurthi et al., 1992, Thromb. Res. 65:177-186; Sakar et al., 1990, Genomics 1990, 6:133-143; and, Katayama et al., 1979, Proc. Natl. Acad. Sci. USA 76:4990-4994.

As set forth herein, heterologous polynucleotide sequences (transgenes) include inhibitory and antisense nucleic acid sequences. Inhibitory, antisense, siRNA (small interfering RNA), miRNA (micro RNA), shRNA (small hairpin RNA), RNAi and antisense oligonucleotides can modulate expression of a target gene. Such molecules include those able to inhibit expression of a target gene involved in mediation of a disease process, thereby reducing, inhibiting or alleviating one or more symptoms of a disease.

Antisense includes single, double or triple stranded polynucleotides and peptide nucleic acids (PNAs) that bind RNA transcript or DNA (e.g., genomic DNA). Oligonucleotides derived from the transcription initiation site of a target gene, e.g., between positions −10 and +10 from the start site, are another particular example. Triplex forming antisense can bind to double strand DNA thereby inhibiting transcription of the gene. “RNAi” is the use of single or double stranded RNA sequences for inhibiting gene expression (see, e.g., Kennerdell et al., Cell 95:1017 (1998); and Fire et al., Nature, 391:806 (1998)). Double stranded RNA sequences from a target gene coding region may therefore be used to inhibit or prevent gene expression/transcription in accordance with the methods and uses of the invention. Antisense and RNAi can be produced based upon nucleic acids encoding target gene sequences (e.g., huntingtin, or HTT), such as nucleic acid encoding mammalian and human HTT. For example, a single or double stranded nucleic acid (e.g., RNA) can target HTT transcript (e.g., mRNA).

A “siRNA” refers to a therapeutic molecule involved in the RNA interference process for a sequence-specific post-transcriptional gene silencing or gene knockdown. siRNAs have homology with the sequence of the cognate mRNA of the targeted gene. Small interfering RNAs (siRNAs) can be synthesized in vitro or generated by ribonuclease III cleavage from longer dsRNA and are the mediators of sequence-specific mRNA degradation. siRNA or other such nucleic acids of the invention can be chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, CA, USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting mRNA of a target gene may be between 15-50 nucleotides in length, and more typically about 20-30 nucleotides in length. Such nucleic acid molecules can be readily incorporated into the viral vectors disclosed herein using conventional methods known to one of skill in the art.

Particular non-limiting examples of genes (e.g., genomic DNA) or transcript of a pathogenic gene (e.g., RNA or mRNA) that may be targeted with inhibitory nucleic acid sequences in accordance with the invention include, but are not limited to: genes associated with polynucleotide repeat diseases such as huntingtin (HTT) gene, a gene associated with dentatorubropallidolusyan atropy (e.g., atrophin 1, ATN1); androgen receptor on the X chromosome in spinobulbar muscular atrophy, human Ataxin-1, -2, -3, and -7, Ca.2.1 P/Q voltage-dependent calcium channel is encoded by the (CACNA1A), TATA-binding protein, Ataxin 8 opposite strand, also known as ATXN8OS, Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercoloesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DAN damage-inducible transcript 4 protein, in diabetic macular edema (DME) or age-related macular degeneration; vascular endothelial growth factor receptor I (VEGFR1) in age-related macular degeneration or choroidal neivascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TORIA) in primary dystonia, pan-class I and HLA-allele specific in transplant; or mutant rhodopsin gene (RHO) in autosomal dominantly inherited retinitis pigmentosa (adRP).

Polynucleotides, polypeptides and subsequences thereof include modified and variant forms. As used herein, the terms “modify” or “variant” and grammatical variations thereof, mean that a polynucleotide, polypeptide or subsequence thereof deviates from a reference sequence. Modified and variant sequences may therefore have substantially the same, greater or less activity or function than a reference sequence, but at least retain partial activity or function of the reference sequence.

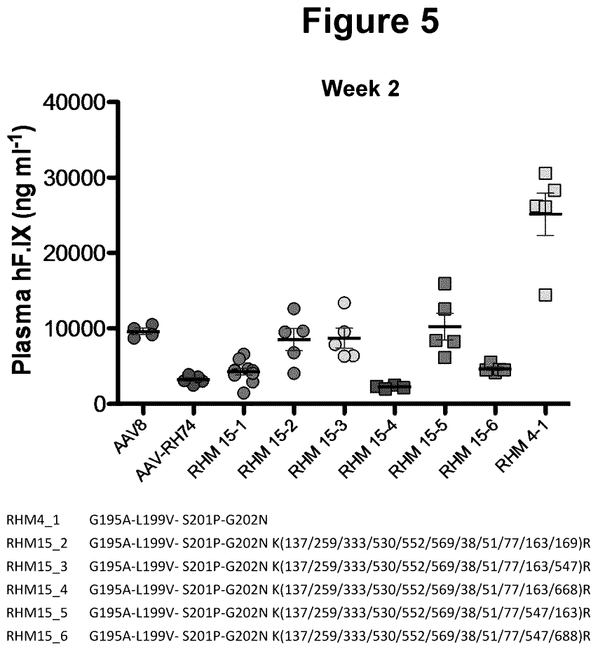

Accordingly, the invention also includes naturally and non-naturally occurring variants. Such variants include AAV-Rh74 variants such as AAV-Rh74 capsid variants. Particular examples of such AAV-Rh74 capsid variants include RHM 15-1, RHM 15-2, RHM 15-3, RHM 15-4, RHM 15-5, RHM 15-6 and RHM4-1 (see, for example, FIG. 5).

Such variants also include gain and loss of function variants. For example, wild type human FIX DNA sequences, which protein variants or mutants retain activity, or are therapeutically effective, or are comparably or even more therapeutically active than invariant human FIX in the methods and uses of the invention. In a particular example, collagen IV serves to trap FIX, meaning that when introduced into the muscle tissue of a mammal some of the FIX is not available for participation in blood coagulation because it is retained in the interstitial spaces in the muscle tissue. A mutation in the sequence of FIX that results in a protein with reduced binding to collagen IV (e.g., loss of function) is a mutant useful in the methods of the invention, for example, for treatment of hemophilia. An example of such a mutant human FIX gene encodes a human FIX protein with the amino acid alanine in place of lysine in the fifth amino acid position from the beginning of the mature protein.

Non-limiting examples of modifications include one or more nucleotide or amino acid substitutions (e.g., 1-3, 3-5, 5-10, 10-15, 15-20, 20-25, 25-30, 30-40, 40-50, 50-100, or more nucleotides or residues), such as an arginine for a lysine residue (e.g., one or more arginine substitution of a lysine as set forth in any of RHM4-1, RHM15-1, RHM15-2, RHM15-3/RHM15-5, RHM15-4 and RHM15-6) additions (e.g., insertions or 1-3, 3-5, 5-10, 10-15, 15-20, 20-25, 25-30, 30-40, 40-50, 50-100, or more nucleotides or residues) and deletions (e.g., subsequences or fragments) of a reference sequence. In particular embodiments, a modified or variant sequence retains at least part of a function or an activity of unmodified sequence. Such modified forms and variants can have less than, the same, or greater, but at least a part of, a function or activity of a reference sequence, for example, as described herein.

A variant can have one or more non-conservative or a conservative amino acid sequence differences or modifications, or both. A “conservative substitution” is the replacement of one amino acid by a biologically, chemically or structurally similar residue. Biologically similar means that the substitution does not destroy a biological activity. Structurally similar means that the amino acids have side chains with similar length, such as alanine, glycine and serine, or a similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine (e.g., RHM15-1, RHM15-2, RHM15-3/RHM15-5, RHM15-4 and RHM15-6), glutamic for aspartic acids, or glutamine for asparagine, serine for threonine, and the like. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. For example, conservative amino acid substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. A “conservative substitution” also includes the use of a substituted amino acid in place of an unsubstituted parent amino acid.

Accordingly, the invention includes gene and protein variants (e.g., of polynucleotides encoding proteins described herein) which retain one or more biological activities (e.g., function in blood clotting, etc.). Such variants of proteins or polypeptides include proteins or polypeptides which have been or may be modified using recombinant DNA technology such that the protein or polypeptide possesses altered or additional properties, for example, variants conferring enhanced protein stability in plasma or enhanced activity of the protein. Variants can differ from a reference sequence, such as naturally occurring polynucleotides, proteins or peptides.

At the nucleotide sequence level, a naturally and non-naturally occurring variant gene will typically be at least about 50% identical, more typically about 70% identical, even more typically about 80% identical (90% or more identity) to the reference gene. At the amino acid sequence level, a naturally and non-naturally occurring variant protein will typically be at least about 70% identical, more typically about 80% identical, even more typically about 90% or more identity to the reference protein, although substantial regions of non-identity are permitted in non-conserved regions (e.g., less, than 70% identical, such as less than 60%, 50% or even 40%). In other embodiments, the sequences have at least 60%, 70%, 75% or more identity (e.g., 80%, 85% 90%, 95%, 96%, 97%, 98%, 99% or more identity) to a reference sequence. Procedures for the introduction of nucleotide and amino acid changes in a polynucleotide, protein or polypeptide are known to the skilled artisan (see. e.g., Sambrook et al., Molecular Cloning: A Laboratory Manual (2007)).

The term “identity,” “homology” and grammatical variations thereof, mean that two or more referenced entities are the same, when they are “aligned” sequences. Thus, by way of example, when two polypeptide sequences are identical, they have the same amino acid sequence, at least within the referenced region or portion. Where two polynucleotide sequences are identical, they have the same polynucleotide sequence, at least within the referenced region or portion. The identity can be over a defined area (region or domain) of the sequence. An “area” or “region” of identity refers to a portion of two or more referenced entities that are the same. Thus, where two protein or nucleic acid sequences are identical over one or more sequence areas or regions they share identity within that region. An “aligned” sequence refers to multiple polynucleotide or protein (amino acid) sequences, often containing corrections for missing or additional bases or amino acids (gaps) as compared to a reference sequence

The identity can extend over the entire sequence length or a portion of the sequence. In particular aspects, the length of the sequence sharing the percent identity is 2, 3, 4, 5 or more contiguous polynucleotide or amino acids, e.g., 6, 7, 8, 9, 10, 11, 12, 13, 14, 15, 16, 17, 18, 19, 20, etc. contiguous amino acids. In additional particular aspects, the length of the sequence sharing identity is 20 or more contiguous polynucleotide or amino acids, e.g., 20, 21, 22, 23, 24, 25, 26, 27, 28, 29, 30, 31, 32, 33, 34, 35, etc. contiguous amino acids. In further particular aspects, the length of the sequence sharing identity is 35 or more contiguous polynucleotide or amino acids, e.g., 35, 36, 37, 38, 39, 40, 41, 42, 43, 44, 45, 45, 47, 48, 49, 50, etc., contiguous amino acids. In yet further particular aspects, the length of the sequence sharing identity is 50 or more contiguous polynucleotide or amino acids, e.g., 50-55, 55-60, 60-65, 65-70, 70-75, 75-80, 80-85, 85-90, 90-95, 95-100, 100-110, etc. contiguous polynucleotide or amino acids.

The terms “homologous” or “homology” mean that two or more referenced entities share at least partial identity over a given region or portion. “Areas, regions or domains” of homology or identity mean that a portion of two or more referenced entities share homology or are the same. Thus, where two sequences are identical over one or more sequence regions they share identity in these regions. “Substantial homology” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology.

The extent of identity (homology) between two sequences can be ascertained using a computer program and mathematical algorithm. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCB1) has exemplary search parameters as follows: Mismatch-2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Polynucleotides include additions and insertions, for example, heterologous domains. An addition (e.g., heterologous domain) can be a covalent or non-covalent attachment of any type of molecule to a composition. Typically additions and insertions (e.g., a heterologous domain) confer a complementary or a distinct function or activity.

Additions and insertions include chimeric and fusion sequences, which is a polynucleotide or protein sequence having one or more molecules not normally present in a reference native (wild type) sequence covalently attached to the sequence. The terms “fusion” or “chimeric” and grammatical variations thereof, when used in reference to a molecule means that a portions or part of the molecule contains a different entity distinct (heterologous) from the molecule as they do not typically exist together in nature. That is, for example, one portion of the fusion or chimera, includes or consists of a portion that does not exist together in nature, and is structurally distinct.

The term “vector” refers to a plasmid, virus (e.g., AAV vector), cosmid, or other vehicle that can be manipulated by insertion or incorporation of a polynucleotide. Such vectors can be used for genetic manipulation (i.e., “cloning vectors”), to introduce/transfer polynucleotides into cells, and to transcribe or translate the inserted polynucleotide in cells. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

As used herein, the term “recombinant,” as a modifier of viral vector, such as recombinant AAV vectors, as well as a modifier of sequences such as recombinant polynucleotides and polypeptides, means that the compositions (e.g., AAV or sequences) have been manipulated (i.e., engineered) in a fashion that generally does not occur in nature. A particular example of a recombinant vector, such as an AAV vector would be where a polynucleotide that is not normally present in the wild-type viral (e.g., AAV) genome is inserted within the viral genome. For example, an example of a recombinant polynucleotide would be where a heterologous polynucleotide (e.g., gene) encoding a protein is cloned into a vector, with or without 5′, 3′ and/or intron regions that the gene is normally associated within the viral (e.g., AAV) genome. Although the term “recombinant” is not always used herein in reference to viral vectors, such as AAV vectors, as well as sequences such as polynucleotides and polypeptides, recombinant forms of AAV, and sequences including polynucleotides and polypeptides, are expressly included in spite of any such omission.

A recombinant vector “genome” (e.g., an AAV vector genome) can be encapsidated or packaged into a virus (also referred to herein as a “particle” or “virion”) for subsequent infection (transduction or transformation) of a cell, ex vivo, in vitro or in vivo. Where a recombinant AAV vector genome is encapsidated or packaged into an AAV particle, the particle can be referred to as a “rAAV.” Such particles or virions will typically include proteins that encapsidate or package the vector genome. Particular examples include viral capsid and envelope proteins, and in the case of AAV, AAV capsid proteins.

For a recombinant plasmid, a vector “genome” refers to the portion of the recombinant plasmid sequence that is ultimately packaged or encapsidated to form a viral particle. In case where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the ‘plasmid backbone,’ which is important for cloning and amplification of the plasmid, a process that is needed for propagation and recombinant virus production, but is not itself packaged or encapsidated into virus (e.g., AAV) particles.

Thus, a vector “genome” refers to the portion of the vector plasmid that is packaged or encapsidated by virus (e.g., AAV), and which contains a heterologous polynucleotide sequence. The non vector genome portion of the recombinant plasmid includes the backbone that is important for cloning and amplification of the plasmid, but is not itself packaged or encapsidated by virus (e.g., AAV).

A viral vector is derived from or based upon one or more nucleic acid elements that comprise a viral genome. Particular viral vectors include parvovirus vectors, such as adeno-associated virus (AAV) vectors.

Recombinant vector sequences are manipulated by insertion or incorporation of a polynucleotide. As disclosed herein, a vector plasmid generally contains at least an origin of replication for propagation in a cell and one or more expression control elements.

Vector sequences including AAV vectors can include one or more “expression control elements.” Typically, expression control elements are nucleic acid sequence(s) that influence expression of an operably linked polynucleotide. Control elements, including expression control elements as set forth herein such as promoters and enhancers, present within a vector are included to facilitate proper heterologous polynucleotide transcription and if appropriate translation (e.g., a promoter, enhancer, splicing signal for introns, maintenance of the correct reading frame of the gene to permit in-frame translation of mRNA and, stop codons etc.). Such elements typically act in cis but may also act in trans.

Expression control can be effected at the level of transcription, translation, splicing, message stability, etc. Typically, an expression control element that modulates transcription is juxtaposed near the 5′ end of the transcribed polynucleotide (i.e., “upstream”). Expression control elements can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, 5000 to 10,000 or more nucleotides from the polynucleotide), even at considerable distances. Nevertheless, owing to the polynucleotide length limitations, for AAV vectors, such expression control elements will typically be within 1 to 1000 nucleotides from the polynucleotide.

Functionally, expression of operably linked heterologous polynucleotide is at least in part controllable by the element (e.g., promoter) such that the element modulates transcription of the polynucleotide and, as appropriate, translation of the transcript. A specific example of an expression control element is a promoter, which is usually located 5′ of the transcribed sequence. Another example of an expression control element is an enhancer, which can be located 5′, 3′ of the transcribed sequence, or within the transcribed sequence.

A “promoter” as used herein can refer to a DNA sequence that is located adjacent to a polynucloetide sequence that encodes a recombinant product. A promoter is typically operatively linked to an adjacent sequence, e.g., heterologous polynucleotide. A promoter typically increases an amount expressed from a heterologous polynucleotide as compared to an amount expressed when no promoter exists.

An “enhancer” as used herein can refer to a sequence that is located adjacent to the heterologous polynucleotide. Enhancer elements are typically located upstream of a promoter element but also function and can be located downstream of or within a DNA sequence (e.g., a heterologous polynucleotide). Hence, an enhancer element can be located 100 base pairs, 200 base pairs, or 300 or more base pairs upstream or downstream of a heterologous polynucleotide. Enhancer elements typically increase expressed of a heterologous polynucleotide above increased expression afforded by a promoter element.

Expression control elements (e.g., promoters) include those active in a particular tissue or cell type, referred to herein as a “tissue-specific expression control elements/promoters.” Tissue-specific expression control elements are typically active in specific cell or tissue (e.g., liver, brain, central nervous system, spinal cord, eye, retina, bone, muscle, lung, pancreas, heart, kidney cell, etc.). Expression control elements are typically active in these cells, tissues or organs because they are recognized by transcriptional activator proteins, or other regulators of transcription, that are unique to a specific cell, tissue or organ type.

For instance, if expression in skeletal muscle is desired, a promoter active in muscle may be used. These include promoters from genes encoding skeletal α-actin, myosin light chain 2A, dystrophin, muscle creatine kinase, as well as synthetic muscle promoters with activities higher than naturally-occurring promoters (see, e.g., Li, et al., Nat. Biotech. 17:241-245 (1999)). Examples of promoters that are tissue-specific for liver are albumin, Miyatake, et al. J. Virol., 71:5124-32 (1997); hepatitis B virus core promoter, Sandig, et al., Gene Ther. 3:1002-9 (1996); alpha-fetoprotein (AFP), Arbuthnot, et al., Hum. Gene. Ther., 7:1503-14 (1996)], bone (osteocalcin, Stein, et al., Mol. Biol. Rep., 24:185-96 (1997); bone sialoprotein, Chen, et al., J. Bone Miner. Res. 11:654-64 (1996)), lymphocytes (CD2, Hansal, et al., J. Immunol., 161:1063-8 (1998); immunoglobulin heavy chain; T cell receptor a chain), neuronal (neuron-specific enolase (NSE) promoter, Andersen, et al., Cell. Mol. Neurobiol., 13:503-15 (1993); neurofilament light-chain gene, Piccioli, et al., Proc. Natl. Acad. Sci. USA, 88:5611-5 (1991); the neuron-specific vgf gene, Piccioli, et al., Neuron, 15:373-84 (1995)]; among others.

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see. e.g., Boshart et al, Cell, 41:521-530 (1985)), the SV40 promoter, the dihydrofolate reductase promoter, the cytoplasmic β-actin promoter and the phosphoglycerol kinase (PGK) promoter.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. A regulatable element that decreases expression of the operably linked polynucleotide in response to a signal or stimuli is referred to as a “repressible element” (i.e., the signal decreases expression such that when the signal, is removed or absent, expression is increased). Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268:1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J. Clin. Invest. 100:2865-2872 (1997); Rivera, et al., Nat. Medicine. 2:1028-1032 (1996)). Other regulatable control elements which may be useful in this context are those which are regulated by a specific physiological state, e.g., temperature, acute phase.

Expression control elements also include the native elements(s) for the heterologous polynucleotide. A native control element (e.g., promoter) may be used when it is desired that expression of the heterologous polynucleotide should mimic the native expression. The native element may be used when expression of the heterologous polynucleotide is to be regulated temporally or developmentally, or in a tissue-specific manner, or in response to specific transcriptional stimuli. Other native expression control elements, such as introns, polyadenylation sites or Kozak consensus sequences may also be used.

As used herein, the term “operable linkage” or “operably linked” refers to a physical or functional juxtaposition of the components so described as to permit them to function in their intended manner. In the example of an expression control element in operable linkage with a polynucleotide, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Vectors including AAV vectors can include still additional nucleic acid elements. These elements include, without limitation one or more copies of an AAV ITR sequence, a promoter/enhancer element, a transcription termination signal, 5′ or 3′ untranslated regions (e.g., polyadenylation sequences) which flank a polynucloetide sequence, or all or a portion of intron I. Such elements also optionally include a transcription termination signal. A particular non-limiting example of a transcription termination signal is the SV40 transcription termination signal.

As disclosed herein, AAV vectors typically accept inserts of DNA having a defined size range which is generally about 4 kb to about 5.2 kb, or slightly more. Thus, for shorter sequences, inclusion of a stuffer or filler in the insert fragment in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. In particular embodiments of an AAV vector, a heterologous polynucleotide sequence has a length less than 4.7 Kb and the filler or stuffer polynucleotide sequence has a length that when combined (e.g., inserted into a vector) with the heterologous polynucleotide sequence has a total length between about 3.0-5.5 Kb, or between about 4.0-5.0 Kb, or between about 4.3-4.8 Kb.

An intron can also function as a filler or stuffer polynucleotide sequence in order to achieve a length for AAV vector packaging into a virus particle. Introns and intron fragments (e.g. portion of intron I of FIX) that function as a filler or stuffer polynucleotide sequence also can enhance expression. For example, inclusion of an intron element may enhance expression compared with expression in the absence of the intron element (Kurachi et al., 1995, supra).

The use of introns is not limited to the inclusion of FIX intron I sequences, but also include other introns, which introns may be associated with the same gene (e.g., where the heterologous polynucleotide encodes FIX, the intron is derived from an intron present in the FIX genomic sequence) or associated with a completely different gene or other DNA sequence. Accordingly, other untranslated (non-protein encoding) regions of nucleic acid, such as introns found in genomic sequences from cognate (related) genes (the heterologous polynucleotide sequence encodes all or a portion of same protein encoded by the genomic sequence) and non-cognate (unrelated) genes (the heterologous polynucleotide sequence encodes a protein that is distinct from the protein encoded by the genomic sequence) can also function as filler or stuffer polynucleotide sequences in accordance with the invention.

A “portion of intron I” as used herein, is meant region of intron I having a nucleotide length of from about 0.1 kb to about 1.7 kb, which region enhances expression of FIX, typically by about 1.5-fold or more on a plasmid or viral vector template when compared with expression of FIX in the absence of a portion of intron I. A more specific portion is a 1.3 kb portion of intron 1.

The term “oligonucleotide” as used herein refers to sequences, primers and probes defined as a nucleic acid molecule comprised of two or more ribo-or deoxyribonucleotides, typically more than three. The exact size of the oligonucleotide will depend on various factors and on the particular application and use of the oligonucleotide, but typically an oligonucleotide has a length between about 5-50 nucleotides.

The term “primer” as used herein refers to a DNA oligonucleotide, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-30 or more nucleotides in length. The primer must be of sufficient complementarity to the desired template to prime the synthesis of the desired extension product, that is, to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188.

The phrase “specifically hybridize” refers to the association between two single-stranded nucleic acid molecules of sufficiently complementary sequence to permit hybridization under predetermined conditions generally used in the art (sometimes termed “substantially complementary”). In particular, the term refers to hybridization of two polynucleotide sequences with substantially complementary sequences, to the substantial exclusion of hybridization with other single-stranded non-complementary nucleic acid sequences.

A “selectable marker gene” refers to a gene that when expressed confers a selectable phenotype, such as antibiotic resistance (e.g., kanamycin), on a transformed cell. A “reporter” gene is one that provides a detectable signal. A non-limiting example of a reporter gene is the luciferase gene.

Polynucleotides and polypeptides including modified forms can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Polynucleotides and polypeptides including modified forms can also be produced by chemical synthesis using methods to the skilled artisan, for example, an automated synthesis apparatus (see, e.g., Applied Biosystems, Foster City, CA). Peptides can be synthesized, whole or in part, using chemical methods (see, e.g., Caruthers (1980). Nucleic Acids Res. Symp. Ser. 215; Horn (1980); and Banga, A. K., Therapeutic Peptides and Proteins, Formulation, Processing and Delivery Systems (1995) Technomic Publishing Co., Lancaster, PA). Peptide synthesis can be performed using various solid phase techniques (see, e.g., Roberge Science 269:202 (1995); Merrifield, Methods Enzymol. 289: 3 (1997)) and automated synthesis may be achieved, e.g., using the ABI 431A Peptide Synthesizer (Perkin Elmer) in accordance with the manufacturer's instructions.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane. The term “isolated” does not exclude combinations produced by the hand of man, for example, a recombinant vector (e.g., AAV) sequence, or virus particle (e.g., AAV-Rh74 vector or related AAV vector such as AAV-Rh74 variants such as capsid variants (e.g., RHM4-1)) that packages or encapsidates a vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

Methods and uses of the invention provide a means for delivering (transducing) heterologous polynucleotides (transgenes) into a broad range of host cells, including both dividing and non-dividing cells. The recombinant vector (e.g., AAV) sequences, plasmids, vector genomes, recombinant virus particles (e.g., AAV-Rh74 or related AAV such as AAV-Rh74 variants such as capsid variants (e.g., RHM4-1)), methods, uses and pharmaceutical formulations of the invention are additionally useful in a method of administering a protein, peptide or nucleic acid to a subject in need thereof, as a method of treatment. In this manner, the protein, peptide or nucleic acid may thus be produced in vivo in a subject. The subject may benefit from or be in need of the protein, peptide or nucleic acid because the subject has a deficiency of the protein, peptide or nucleic acid, or because the production of the protein, peptide or nucleic acid in the subject may impart some therapeutic effect, as a method of treatment or otherwise. Alternatively, it may be desirable to inhibit or reduce expression or production of a target gene involved in a disease process, e.g., for the treatment of a neurodegenerative disease, cancer or atherosclerosis, for example to achieve a therapeutic effect.

In general, recombinant vector (e.g., AAV) sequences, plasmids, vector genomes, recombinant virus particles (e.g., AAV-Rh74 or related AAV such as AAV-Rh74 variants such as capsid variants (e.g., RHM4-1)), methods and uses may be used to deliver any heterologous polynucleotide (transgene) with a biological effect to treat or ameliorate one or more symptoms associated with any disorder related to insufficient or undesirable gene expression. Recombinant vector (e.g., AAV) sequences, plasmids, vector genomes, recombinant virus particles (e.g., AAV-Rh74 or related AAV such as AAV-Rh74 variants such as capsid variants (e.g., RHM4-1)) particles, methods and uses may be used to provide therapy for various disease states.

There are a number of inherited diseases in which defective genes are known and have been cloned. In general, the above disease states fall into two classes: deficiency states, usually of enzymes, which are generally inherited in a recessive manner, and unbalanced states, at least sometimes involving regulatory or structural proteins, which are inherited in a dominant manner. For deficiency state diseases, gene transfer could be used to bring a normal gene into affected tissues for replacement therapy, as well as to create animal models for the disease using antisense mutations. For unbalanced disease states, gene transfer could be used to create a disease state in a model system, which could then be used in efforts to counteract the disease state. Thus, recombinant vector (e.g., AAV) sequences, plasmids, vector genomes, recombinant virus particles (e.g., AAV-Rh74 vectors or related AAV vectors such as AAV-Rh74 variants such as capsid variants (e.g., RHM4-1)), methods and uses permit the treatment of genetic diseases. As used herein, a disease state is treated by partially or wholly remedying the deficiency or imbalance that causes the disease or makes it more severe. The use of site-specific integration of nucleic acid sequences to cause mutations or to correct defects is also possible.

Illustrative disease states include, but are not limited to: cystic fibrosis (and other diseases of the lung), hemophilia A, hemophilia B, thalassemia, anemia and other blood coagulation disorders, AIDs, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Gaucher's disease, Hurler's disease, adenosine deaminase deficiency, glycogen storage diseases and other metabolic defects, Pompe's disease, congestive heart failure, retinal degenerative diseases (choroideremia, Leber's congenital amaurosis, and other diseases of the eye), diseases of solid organs (e.g., brain, liver, kidney, heart), and the like.

In accordance with the invention, treatment methods and uses are provided that include invention recombinant vectors (e.g., AAV), vector genomes, recombinant virus particles (e.g., AAV-Rh74 vectors or related AAV vectors such as AAV-Rh74 variants such as capsid variants (e.g., RHM4-1)) and invention viral particles (e.g., AAV-Rh74 or related AAV such as AAV-Rh74 variants such as capsid variants (e.g., RHM4-1)) including vector genomes. Methods and uses of the invention are broadly applicable to diseases amenable to treatment by introducing a gene encoding a protein, or increasing or stimulating gene expression or function, e.g., gene addition or replacement. Methods and uses of the invention are also broadly applicable to diseases amenable to treatment by reducing or decreasing gene expression or function, e.g., gene knockout or reduction of gene expression (gene knockdown).

Non-limiting particular examples of diseases treatable in accordance with the invention include those set forth herein as well as a lung disease (e.g., cystic fibrosis), a blood coagulation or bleeding disorder (e.g., hemophilia A or hemophilia B with or without inhibitors), thalassemia, a blood disorder (e.g., anemia), Alzheimer's disease, Parkinson's disease, Huntington's disease, amyotrophic lateral sclerosis (ALS), epilepsy, lysosomal storage diseases, a copper or iron accumulation disorders (e.g., Wilson's or Menkes disease) lysosomal acid lipase deficiency, a neurological or neurodegenerative disorder, cancer, type 1 or type 2 diabetes, Gaucher's disease, Hurler's disease, adenosine deaminase deficiency, a metabolic defect (e.g., glycogen storage diseases), a retinal degenerative disease (such as RPE65 deficiency or defect, choroideremia, and other diseases of the eye), and a disease of a solid organ (e.g., brain, liver, kidney, heart).

In addition, invention recombinant vectors (e.g., AAV), vector genomes, recombinant virus particles (e.g., AAV-Rh74 vectors or related AAV vectors such as AAV-Rh74 variants such as capsid variants (e.g., RHM4-1)), methods and uses may be employed to deliver nucleic acids encoding monoclonal antibodies or fragments thereof to provide beneficial biological effects to treat or ameliorate the symptoms associated with cancers, infectious diseases, and autoimmune diseases such as rheumatoid arthritis.

In one embodiment, a method or use of the invention includes: (a) providing a viral particle (e.g., AAV-Rh74 or related AAV such as AAV-Rh74 capsid variants (e.g., RHM4-1)) comprising a vector genome, the vector genome comprising a heterologous polynucleotide sequence (and, optionally a filler/stuffer polynucleotide sequence), wherein the heterologous polynucleotide sequence is operably linked to an expression control element conferring transcription of said polynucleotide sequence; and (b) administering an amount of the viral particle to the mammal such that said heterologous polynucleotide is expressed in the mammal.

In another embodiment, a method or use of the invention includes delivering or transferring a heterologous polynucleotide sequence into a mammal or a cell of a mammal, by administering a viral (e.g., AAV) particle (e.g., AAV-Rh74 or related AAV such as AAV-Rh74 variants such as capsid variants (e.g., RHM4-1)) or plurality of viral (e.g., AAV) particles (e.g., AAV-Rh74 or related AAV such as AAV-Rh74 variants such as capsid variants (e.g., RHM4-1)) comprising a vector genome, the vector genome comprising the heterologous polynucleotide sequence (and optionally a filler/stuffer polynucleotide sequence) to a mammal or a cell of a mammal, thereby delivering or transferring the heterologous polynucleotide sequence into the mammal or cell of the mammal.

In a further embodiment, a method or use of the invention for treating a mammal deficient in need of protein expression or function includes providing a viral (e.g., AAV) particle (e.g., AAV-Rh74 or related AAV such as AAV-Rh74 variants such as capsid variants (e.g., RHM4-1)) or plurality of viral (e.g., AAV) particles (e.g., AAV-Rh74 or related AAV such as AAV-Rh74 variants such as capsid variants (e.g., RHM4-1)) comprising a vector genome, the vector genome comprising a heterologous polynucleotide sequence (and optionally a filler/stuffer polynucleotide sequence); and administering the viral particle or plurality of viral particles to the mammal, where the heterologous polynucleotide sequence encodes a protein expressed in the mammal, or where the heterologous polynucleotide sequence encodes an inhibitory sequence or protein that reduces expression of an endogenous protein in the mammal.

In particular aspects of invention methods and uses disclosed herein, expression of the heterologous polynucleotide encodes a protein or inhibitory nucleic acid that provides a therapeutic benefit to the mammal (e.g., human). In further particular aspects, a filler/stuffer polynucleotide sequence is included in the vector sequence such that the combined length with the heterologous polynucleotide sequence has a total length of between about 3.0-5.5 Kb, or between about 4.0-5.0 Kb, or between about 4.3-4.8 Kb.

Methods and uses of the invention include treatment methods, which result in any therapeutic or beneficial effect. In various invention methods and uses, further included are inhibiting, decreasing or reducing one or more adverse (e.g., physical) symptoms, disorders, illnesses, diseases or complications caused by or associated with the disease, such as reduced blood clotting time, reduced administration dosage of supplemental clotting factor protein.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, or complication of a disease. Thus, a satisfactory clinical endpoint is achieved when there is an incremental improvement or a partial reduction in an adverse symptom, disorder, illness, or complication caused by or associated with a disease, or an inhibition, decrease, reduction, suppression, prevention, limit or control of worsening or progression of one or more adverse symptoms, disorders, illnesses, or complications caused by or associated with the disease, over a short or long duration (hours, days, weeks, months, etc.).

Compositions, such as vector genomes, recombinant virus particles (e.g., AAV-Rh74 vectors or related AAV vectors such as AAV-Rh74 variants such as capsid variants (e.g., RHM4-1)) including vector genomes, and methods and uses of the invention, can be administered in a sufficient or effective amount to a subject in need thereof. An “effective amount” or “sufficient amount” refers to an amount that provides, in single or multiple doses, alone or in combination, with one or more other compositions (therapeutic agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

The vector genome or virus particle (e.g., AAV, such as AAV-Rh74 vector or related AAV vector such as AAV-Rh74 variants such as capsid variants (e.g., RHM4-1)) dose to achieve a therapeutic effect, e.g., the dose in vector genomes/per kilogram of body weight (vg/kg), will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (protein), and the stability of the protein expressed. One skilled in the art can readily determine a virion dose range to treat a patient having a particular disease or disorder based on the aforementioned factors, as well as other factors. Generally, doses will range from at least 1×10⁸, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect.

Using hemophilia as an example, generally speaking, it is believed that, in order to achieve a therapeutic effect, a blood coagulation factor concentration that is greater than 1% of factor concentration found in a normal individual is needed to change a severe disease phenotype to a moderate one. A severe phenotype is characterized by joint damage and life-threatening bleeds. To convert a moderate disease phenotype into a mild one, it is believed that a blood coagulation factor concentration greater than 5% of normal is needed. With respect to treating such a hemophilic subject, a typical dose is at least 1×10¹⁰ vector genomes (vg) per kilogram (vg/kg) of the weight of the subject, or between about 1×10¹⁰ to 1×10¹¹ vg/kg of the weight of the subject, or between about 1×10¹¹ to 1×10¹² vg/kg of the weight of the subject, or between about 1×10¹² to 1×10¹³ vg/kg of the weight of the subject, to achieve a desired therapeutic effect.

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol, such as administration of recombinant clotting factor protein for treatment of a clotting disorder (e.g., hemophilia A or B).

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use. Thus, appropriate amounts will depend upon the condition treated, the therapeutic effect desired, as well as the individual subject (e.g., the bioavailability within the subject, gender, age, etc.).

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease.

Thus, a successful treatment outcome can lead to a “therapeutic effect,” or “benefit” of decreasing, reducing, inhibiting, suppressing, limiting, controlling or preventing the occurrence, frequency, severity, progression, or duration of a disease, or one or more adverse symptoms or underlying causes or consequences of the disease in a subject. Treatment methods and uses affecting one or more underlying causes of the disease or adverse symptoms are therefore considered to be beneficial. A decrease or reduction in worsening, such as stabilizing the disease, or an adverse symptom thereof, is also a successful treatment outcome.

A therapeutic benefit or improvement therefore need not be complete ablation of the disease, or any one, most or all adverse symptoms, complications, consequences or underlying causes associated with the disease. Thus, a satisfactory endpoint is achieved when there is an incremental improvement in a subject's disease, or a partial decrease, reduction, inhibition, suppression, limit, control or prevention in the occurrence, frequency, severity, progression, or duration, or inhibition or reversal, of the disease (e.g., stabilizing one or more symptoms or complications), over a short or long duration of time (hours, days, weeks, months, etc.). Effectiveness of a method or use, such as a treatment that provides a potential therapeutic benefit or improvement of a disease, can be ascertained by various methods.

Invention methods and uses can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents and drugs. Such biologics (proteins), agents, drugs, treatments and therapies can be administered or performed prior to, substantially contemporaneously with or following any other method or use of the invention, for example, a therapeutic method of treating a subject for a blood clotting disease.

The compound, agent, drug, treatment or other therapeutic regimen or protocol can be administered as a combination composition, or administered separately, such as concurrently or in series or sequentially (prior to or following) delivery or administration of a vector genome or virus (e.g., AAV-Rh74 vector or related AAV vector such as AAV-Rh74 variants such as capsid variants (e.g., RHM4-1)) of the invention. The invention therefore provides combinations in which a method or use of the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a vector genome or virus (e.g., AAV-Rh74 vector or related AAV vector such as AAV-Rh74 variants such as capsid variants (e.g., RHM4-1)) of the invention, to a subject. Specific non-limiting examples of combination embodiments therefore include the foregoing or other compound, agent, drug, therapeutic regimen, treatment protocol, process, remedy or composition.

Methods and uses of the invention also include, among other things, methods and uses that result in a reduced need or use of another compound, agent, drug, therapeutic regimen, treatment protocol, process, or remedy. For example, for a blood clotting disease, a method or use of the invention has a therapeutic benefit if in a given subject a less frequent or reduced dose or elimination of administration of a recombinant clotting factor protein to supplement for the deficient or defective (abnormal or mutant) endogenous clotting factor in the subject. Thus, in accordance with the invention, methods and uses of reducing need or use of another treatment or therapy are provided.

The invention is useful in animals including veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of blood clotting diseases and others known to those of skill in the art.

As set forth herein, vectors and virus particles (e.g., AAV-Rh74 or related AAV such as AAV-Rh74 variants such as capsid variants (e.g., RHM4-1)) comprising such vectors can be used to provide a protein to a subject where there is an insufficient amount of the protein or a deficiency in a functional gene product (protein), or to provide an inhibitory nucleic acid or protein to a subject who produces an aberrant, partially functional or non-functional gene product (protein) which can lead to disease. Accordingly, subjects appropriate for treatment include those having or at risk of producing an insufficient amount or having a deficiency in a functional gene product (protein), or produce an aberrant, partially functional or non-functional gene product (protein), which can lead to disease. Subjects appropriate for treatment in accordance with the invention also include those having or at risk of producing an aberrant, or defective (mutant) gene product (protein) that leads to a disease such that reducing amounts, expression or function of the aberrant, or defective (mutant) gene product (protein) would lead to treatment of the disease, or reduce one or more symptoms or ameliorate the disease. Target subjects therefore include subjects that have such defects regardless of the disease type, timing or degree of onset, progression, severity, frequency, or type or duration of symptoms.

“Prophylaxis” and grammatical variations thereof mean a method in which contact, administration or in vivo delivery to a subject is prior to disease. Administration or in vivo delivery to a subject can be performed prior to development of an adverse symptom, condition, complication, etc. caused by or associated with the disease. For example, a screen (e.g., genetic) can be used to identify such subjects as candidates for invention methods and uses, but the subject may not manifest the disease. Such subjects therefore include those screened positive for an insufficient amount or a deficiency in a functional gene product (protein), or that produce an aberrant, partially functional or non-functional gene product (protein), which can lead to disease; and subjects that screen positive for an aberrant, or defective (mutant) gene product (protein) that leads to disease, even though such subjects do not manifest symptoms of the disease.

Methods and uses of the invention include delivery and administration systemically, regionally or locally, or by any route, for example, by injection, infusion, orally (e.g., ingestion or inhalation), or topically (e.g., transdermally). Such delivery and administration include intravenously, intramuscularly, intraperitoneally, intradermally, subcutaneously, intracavity, intracranially, transdermally (topical), parenterally, e.g. transmucosally or rectally. Exemplary administration and delivery routes include intravenous (i.v.), intraperitoneal (i.p.), intrarterial, intramuscular, parenteral, subcutaneous, intra-pleural, topical, dermal, intradermal, transdermal, parenterally, e.g. transmucosal, intra-cranial, intra-spinal, oral (alimentary), mucosal, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, intralymphatic.

Doses can vary and depend upon whether the treatment is prophylactic or therapeutic, the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

Methods and uses of the invention as disclosed herein can be practiced within 1-2, 2-4, 4-12, 12-24 or 24-72 hours after a subject has been identified as having the disease targeted for treatment, has one or more symptoms of the disease, or has been screened and is identified as positive as set forth herein even though the subject does not have one or more symptoms of the disease. Of course, methods and uses of the invention can be practiced 1-7, 7-14, 14-21, 21-48 or more days, months or years after a subject has been identified as having the disease targeted for treatment, has one or more symptoms of the disease, or has been screened and is identified as positive as set forth herein.

Recombinant vector (e.g., AAV, such as AAV-Rh74 vector or related AAV vector such as AAV-Rh74 variants such as capsid variants (e.g., RHM4-1)), sequences, plasmids, vector genomes, recombinant virus particles (e.g., AAV, such as AAV-Rh74 vector or related AAV vector such as AAV-Rh74 variants such as capsid variants (e.g., RHM4-1)) and other compositions, agents, drugs, biologics (proteins) can be incorporated into pharmaceutical compositions, e.g., a pharmaceutically acceptable carrier or excipient. Such pharmaceutical compositions are useful for, among other things, administration and delivery to a subject in vivo or ex vivo.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a viral vector or viral particle (e.g., AAV-Rh74 or related AAV such as AAV-Rh74 variants such as capsid variants (e.g., RHM4-1)) or transformed cell to a subject.

Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Carriers which may be used include Vaseline, lanolin, polyethylene glycols, alcohols, transdermal enhancers, and combinations thereof.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Pharmaceutical compositions and delivery systems appropriate for the vector genomes, virus particles (e.g., AAV-Rh74 vector or related AAV vector such as AAV-Rh74 variants such as capsid variants (e.g., RHM4-1)) and methods and uses of the invention are known in the art (see, e.g., Remington: The Science and Practice of Pharmacy (2003) 20^{th} ed., Mack Publishing Co., Easton, PA; Remington's Pharmaceutical Sciences (1990) 18^{th} ed., Mack Publishing Co., Easton, PA; The Merck Index (1996) 12^{th} ed., Merck Publishing Group, Whitehouse, NJ; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, MD; and Poznansky et al., Drug Delivery Systems (1980), R. L. Juliano, ed., Oxford, N.Y., pp. 253-315).

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Recombinant vector (e.g., AAV) sequences, plasmids, vector genomes, recombinant virus particles (e.g., AAV-Rh74 vector or related AAV vector such as AAV-Rh74 capsid variants (e.g., RHM4-1)), and pharmaceutical compositions thereof can be packaged in single or multiple unit dosage form for ease of administration and uniformity of dosage.

The invention provides kits with packaging material and one or more components therein. A kit typically includes a label or packaging insert including a description of the components or instructions for use in vitro, in vivo, or ex vivo, of the components therein. A kit can contain a collection of such components, e.g., a vector (e.g., AAV) genome or virus particle (e.g., AAV-Rh74 vector or related AAV vector such as AAV-Rh74 variants such as capsid variants (e.g., RHM4-1)) and optionally a second active, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD-or DVD-ROM/RAM, DVD, MP3, magnetic tape, or an electrical storage media such as RAM and ROM or hybrids of these such as magnetic/optical storage media, FLASH media or memory type cards.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., a recombinant vector (e.g., AAV) sequence, plasmid, vector genome, or recombinant virus particle (e.g., AAV-Rh74 vector or related AAV vector such as AAV-Rh74 capsid variants (e.g., RHM4-1)) are an example of a genus of equivalent or similar features.

As used herein, the singular forms “a”, “and,” and “the” include plural referents unless the context clearly indicates otherwise. Thus, for example, reference to “a polynucleotide” includes a plurality of such polynucleotides, reference to “a vector” includes a plurality of such vectors, and reference to “a virus” or “particle” includes a plurality of such virions/particles.

As used herein, all numerical values or numerical ranges include integers within such ranges and fractions of the values or the integers within ranges unless the context clearly indicates otherwise. Thus, to illustrate, reference to at least 80% identity, includes 81%, 82%, 83%, 84%, 85%, 86%, 87%, 88%, 89%, 90%, 91%, etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 1,000, includes 999, 998, 997, etc. all the way down to the number one (1); and less than 100, includes 99, 98, 97, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as a percentage range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17,18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges of 11-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### EXAMPLES

### Example 1

### This Example Includes a Description of Various Materials and Methods.

Mice: Male C57BL/6J (WT) mice 8-10 weeks of age, n=5 per experimental group. The dog is a HB dog from the University of North Carolina Chapel Hill colony carrying a missense mutation in the FIX gene (Evans et al., Proc Natl Acad Sci USA 86:10095 (1989)).

AAV Vector Constructs: The in vivo studies in mice were performed using a construct expressing human FIX under the control of the ApoE-hAAT liver specific promoter. The study in dogs used a nearly identical promoter and the canine FIX transgene.

Gene Transfer Methodology: All vectors were delivered intravenously. In mice via the tail vein (a volume of 200 microliters per mouse was administered, vector was diluted in PBS). In dogs the vector was delivered via the saphenous vein.

FIX Expression Determination: ELISA was used to measure FIX levels. In mice, the human FIX ELISA antibody pair (capture and secondary) is from Affinity Biologicals. In dogs, an antibody pair also from Affinity Biologicals was used as described in Haurigot et al. (Mol Ther 18:1318 (2010)).

Statistical analysis: Statistical analysis was performed with unpaired, two tailed t test. p values <0.05 were considered statistically significant.

AAV Antibody Measurements: An in vitro neutralization assay described in Manno et al., (Nat Med 12:342 (2006)) and Mingozzi et al. (Nat Med 13:419 (2007)) was used for antibody measurement. In brief, two AAV vector constructs were used in the assay, a single-stranded vector expressing β-galactosidase under the control of the CMV promoter (ssAAV-LacZ), or a self-complementary vector expressing the Renilla reporter gene, AAV-Rh74-CBA-Renilla, under the control of the chicken β-actin promoter (CBA). To increase the efficiency of transduction of AAV vectors in vitro, 2V6.11 cells (ATCC) were used, which expressed the adenoviral gene E4 under the control of an inducible promoter. Cells were seeded in a 96-well plate at a density of 1.25×10⁴ cells/well and a 1:1000 dilution of ponasterone A (Invitrogen) was added to the medium to induce E4 expression. At the day of assay, serial half-log dilutions of heat-inactivated test serum were mixed with medium containing virus. For the ssAAV-LacZ vector, virus concentration used in the assay was ˜1×10¹⁰ vg/ml for AAV2 and ˜5.5×10¹⁰ vg/ml for AAV5, 6, or 8. For the scAAV-Luc vector, virus concentration in the assay was between ˜50 and 150-fold lower. Residual activity of the reporter transgene was measured using either a colorimetric assay (ssAAV-LacZ) or a luminometer (scAAV-Luc).

Anti-AAV capsid total IgG or Ig subclasses were measured with a capture assay; ELISA plates were coated with 5×10¹⁰ capsid particles/ml of AAV empty capsids. Plates were blocked with 2% BSA, 0.05% Tween 20 in PBS for 2 hours at room temperature and serial dilutions of samples were loaded onto the wells and incubated overnight at 4° C. Biotin-conjugated anti-human IgG1, IgG2, IgG3, IgG4, or IgM antibody (Sigma) were used as detecting antibodies; streptavidin-HRP was the added for substrate detection. Ig concentration was determined against standard curves made with serial dilution of human purified IgG1, IgG2, IgG3, IgG4, or IgM (Sigma).

AAV Production: The process for vector production is described in detail in Ayuso et al. (Gene Ther 17:503 (2010)).

### Example 2

### This Example Includes a Description of Human FIX Gene Transfer Animal (Mice) Studies and FIX Expression After Gene Transfer.

C57BL/6 mice (n=5 per group) were injected via the tail vein with AAV vectors bearing the Factor IX (FIX) gene (2.5¹⁰ vector genomes per mouse) under the control of a liver-specific promoter. Human FIX transgene product (protein) plasma levels in the mice were determined by ELISA at week 1, 2, and 4 post gene transfer, and are illustrated in FIG. 1. AAV-Rh74 showed the highest level of transgene expression in the animals.

### Example 3

### This Example Includes a Description of Animal Studies and Data Demonstrating Effective AAV-Rh74 Mediated Delivery of FIX at Therapeutic Levels in Hemophilia Dogs.

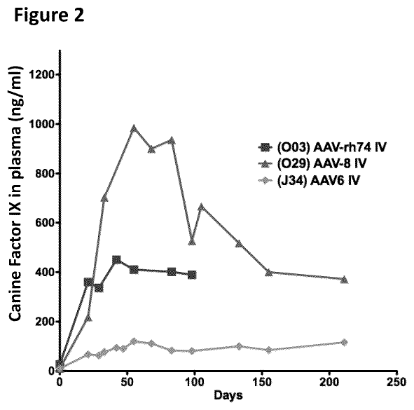

In brief, hemophilia B dogs were infused intravenously (IV) though the saphenous vein with 3×10¹² vector genomes per kg of weight. Expression of the therapeutic FIX transgene was driven by a liver specific promoter. Vectors and FIX levels were monitored by ELISA. Canine FIX plasma levels are shown in FIG. 2. AAV-Rh74 and AAV8 performed roughly equally in hemophilia B dogs, and both were superior to AAV6.

### Example 4

### This Example Includes a Description of Studies Showing the Presence of Anti-AAV Neutralizing Antibodies (NAb) in Humans.

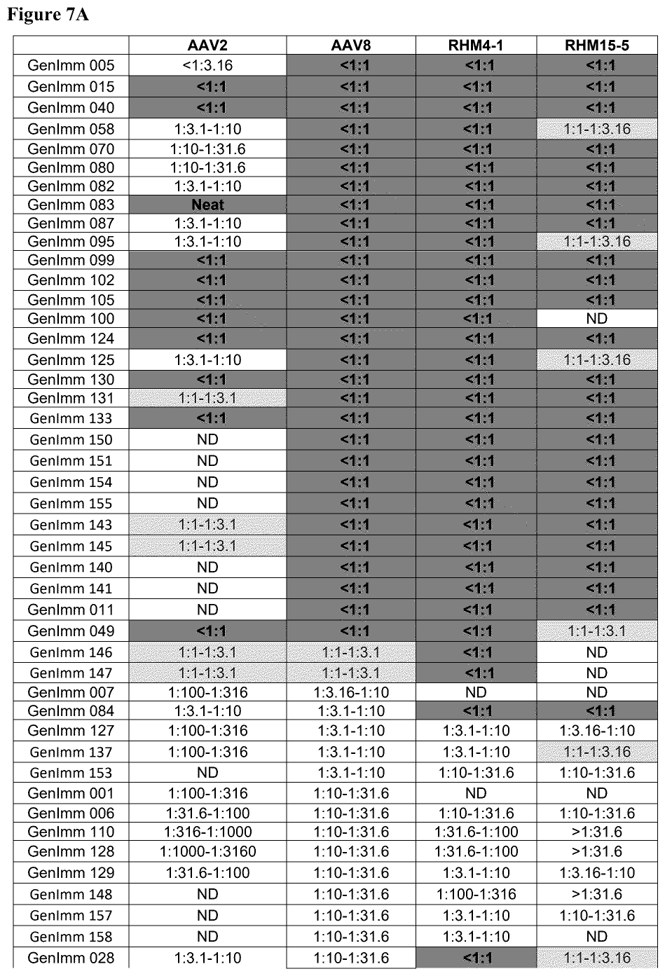

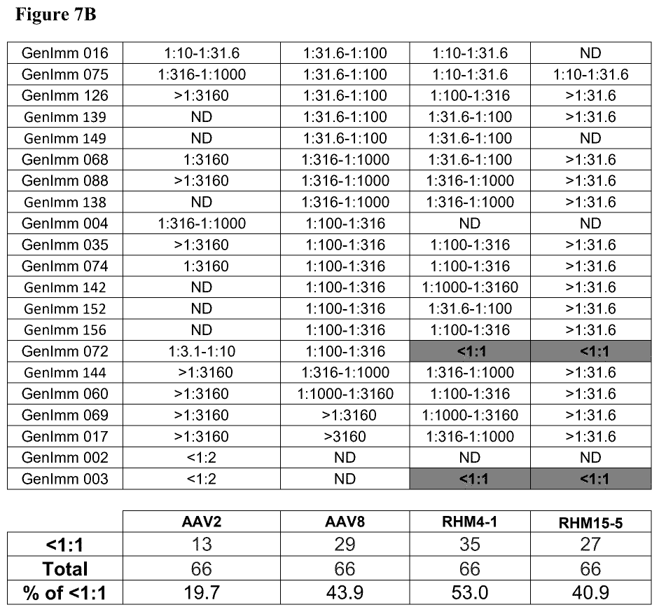

The data in FIG. 7 show anti-AAV neutralizing antibodies (NAb) measured in humans with an in vitro assay. Subjects with a NAb titer of less than or equal to 1:1 are defined as naïve or low-titer for anti-AAV antibodies, and are eligible for gene transfer for that AAV serotype (highlighted in grey). Patients with titers between 1:1 and 1:3 are considered AAV-permissive as long as empty capsids are used as decoys. Samples with titers higher than 1:3 are considered non-permissive to AAV transduction after systemic injection and are filled in light pink. AAV-Rh74 exhibited the lowest prevalence of anti-AAV Nab compared to AAV-2 and AAV-8.

### Example 5

### This Example Includes a Description of Data Showing Production Amounts of Different AAV Serotypes Including AAV-Rh74.

The data in Table 1 show production yield of different AAV serotypes. Reported are the virus batch size in roller bottles, the total vector yield, and the yield per bottle. All serotypes were packaged with the same expression cassette. AAV-Rh74 has a yield comparable to or greater than the other serotypes evaluated, namely AAV-8, AAV-dj, and AAV-2.

TABLE 1

| | Batch size | Total vector | Yield per |
|---|---|---|---|
| | (number of | yield | roller bottle |
| Serotype | roller bottles) | (vector genomes) | (vector genomes) |
| AAV-Rh74 | 80 | 1.21E+15 | 1.51E+13 |
| AAV-Rh74 | 10 | 1.23E+14 | 1.23E+13 |
| AAV-8 | 30 | 2.54E+14 | 8.47E+12 |
| AAV-dj | 20 | 1.79E+14 | 8.95E+12 |
| AAV-2 | 30 | 1.38E+14 | 4.60E+12 |

### Example 6

This Example Includes a Description of Data Showing that AAVrh74 Vector Expressing Human Factor IX (FIX) Under the Control of a Liver-Specific Promoter Administered to Rhesus Macaques Led to Production Amounts of FIX in Animals, and at Higher Levels than AAV8 Vector Administered at the Same Amount.

In brief, animals were administered either AAV8 or AAVrh74 at a dose of 2×10¹² vector genomes (vg)/kg of weight. Vectors were either formulated in saline or in a mixture of vector and empty AAV capsid (denoted EC).

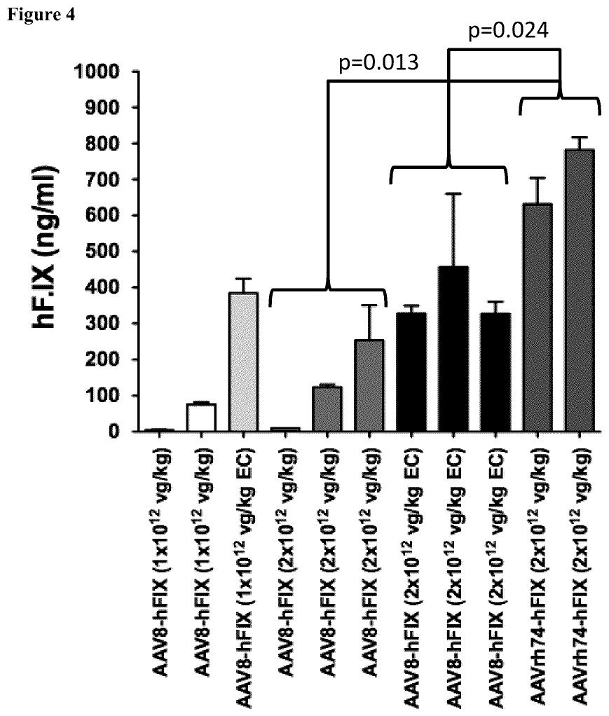

FIG. 4 is a histogram plot of the average (weeks 2 to 8) and standard error or the mean of human FIX measured by an ELISA that detects specifically human FIX in rhesus macaque plasma. Animals receiving the AAVrh74-FIX vector are shown in the last two bars towards the right margin. The data shows that animals receiving the AAVrh74 vectors (last two bars towards right margin) expressed the FIX transgene at higher levels compared to the other groups of animals injected at the same dose (black and grey bars). Average levels were compared using unpaired, two-tailed student t test.

One of the animals receiving AAV-RHM4-1-FIX developed an inhibitor against the human factor IX transgene product, which is a well-documented phenomenon that occurs in approximately 20% of macaques treated with a human FIX vector. The second RHM4-1-treated animal expressed FIX levels around 2-fold higher compared to the AAV8-treated macaques.

### Example 7

### This Example Includes a Description of Several Rh74 Capsid Variants.

In brief, various substitutions were introduced into Rh74 capsid sequence to produce Rh74 capsid variants. The different Rh74 capsid variants, and substituted amino acids at each position, were as follows:

TABLE 2

| | Amino Acid Substitutions and |
|---|---|
| Variants | Indicated Positions in VP1 Capsid |
| RHM4_1 | G195A-L199V- S201P-G202N |
| RHM15_1 | G195A-L199V-S201P-G202N |
| | K(137/259/333/530/552/569/38/51/77/169/547)R |
| RHM15_2 | G195A-L199V- S201P-G202N |
| | K(137/259/333/530/552/569/38/51/77/163/169)R |
| RHM15_3 | G195A-L199V- S201P-G202N |
| | K(137/259/333/530/552/569/38/51/77/163/547)R |
| RHM15_4 | G195A-L199V- S201P-G202N |
| | K(137/259/333/530/552/569/38/51/77/163/668)R |
| RHM15_5 | G195A-L199V- S201P-G202N |
| | K(137/259/333/530/552/569/38/51/77/547/163)R |
| RHM15_6 | G195A-L199V- S201P-G202N |
| | K(137/259/333/530/552/569/38/51/77/547/688)R |

RHM4-1 variant had an alanine, a valine, a proline, and an asparagine substitution at amino acid positions 195, 199, 201 and 202, respectively, of Rh74 VP1 capsid. The RHM4-1 variant VP1 capsid amino acid sequence, with substituted residues a, v, p and n, underlined and in bold, is as follows (SEQ ID NO:5):

| | |
|---|---|
| 1 | maadgylpdwlednlsegirewwdlkpgapkpkanqqkqdngrglvlpgykylgpfngld |
| 61 | kgepvnaadaaalehdkaydqqlqagdnpylrynhadaefgerlqedtsfggnlgravfq |
| 121 | akkrvleplglvespvktapgkkrpvepspqrspdsstgigkkgqqpakkrinfgqtgds |
| 181 | esvpdpqpigeppaapsgvgpntmaagggapmadnnegadgvgsssgnwhcdstwlgdry |
| 241 | ittstrtwalptynnhlykqisngtsggstndntyfgystpwgyfdfnrfhchfsprdwq |
| 301 | rlinnnwgfrpkrinfklfniqvkevtqnegtktiannitstiqvftdseyqlpyvlgsa |
| 361 | hqgclppfpadvfmipqygyltlnngsqav grssfycleyfpsqmlrtgnnfefsynfed |
| 421 | vpfhssyahsqsldrlmnplidqylyylsrtqstggtagtqqllfsqagpnnmsaqaknw |
| 481 | 1pgpcyrqqrvsttlsqnnnsnfawtgatkyhlngrdslvnpgvamathkddeerffpss |
| 541 | gvlmfgkqgagkdnvdyssvmltseeeikttnpvateqygvvadnlqqqnaapivgavns |
| 601 | qgalpgmvwqnrdvylqgpiwakiphtdgnfhpsplmggfglkhpppgilikntpvpadp |
| 661 | pttfnqaklasfitqystgqvsveiewelqkenskrwnpeiqytsnyykstnvdfavnte |
| 721 | gtyseprpigtryltrnl |

The RHM4-1 variant VP1 capsid nucleic acid sequence, with codons encoding a, v, p and n. underlined and in bold, is as follows (SEQ ID NO:11):

| | |
|---|---|
| 1 | atggctgccgatggttatcttccagattggctcgaggacaacctctctgagggcattcgc |
| 61 | gagtggtgggacctgaaacctggagccccgaaacccaaagccaaccagcaaaagcaggac |
| 121 | aacggccggggtctggtgcttcctggctacaagtacctcggacccttcaacggactcgac |
| 181 | aagggggagcccgtcaacgcggcggacgcagcggccctcgagcacgacaaggcctacgac |
| 241 | cagcagctccaagcgggtgacaatccgtacctgcggtataatcacgccgacgccgagttt |
| 301 | caggagcgtctgcaagaagatacgtcttttgggggcaacctcgggcgcgcagtcttccag |
| 361 | gccaaaaagcgggttctcgaacctctgggcctggttgaatcgccggttaagacggctcct |
| 421 | ggaaagaagagaccggtagagccatcaccccagcgctctccagactcctctacgggcatc |
| 481 | ggcaagaaaggccagcagcccgcaaaaaagagactcaattttgggcagactggcgactca |
| 541 | gagtcagtccccgaccctcaaccaatcggagaaccaccagcagccccctctggtqtqgga |
| 601 | cctaatacaatggctgcaggcggtggcgctccaatggcagacaataacgaaggcgccgac |
| 661 | ggagtgggtagttcctcaggaaattggcattgcgattccacatggctgggcgacagagtc |
| 721 | atcaccaccagcacccgcacctgggccctgcccacctacaacaaccacctctacaagcaa |
| 781 | atctccaacgggacctcgggaggaagcaccaacgacaacacctacttcggctacagcacc |
| 841 | ccctgggggtattttgacttcaacagattccactgccacttttcaccacgtgactggcag |
| 901 | cgactcatcaacaacaactggggattccggcccaagaggctcaacttcaagctcttcaac |
| 961 | atccaagtcaaggaggtcacgcagaatgaaggcaccaagaccatcgccaataaccttacc |
| 1021 | agcacgattcaggtctttacggactcggaataccagctcccgtacgtgctcggctcggcg |
| 1081 | caccagggctgcctgcctccgttcccggcggacgtcttcatgattcctcagtacgggtac |
| 1141 | ctgactctgaacaatggcagtcaggctgtgggccggtcgtccttctactgcctggagtac |
| 1201 | tttccttctcaaatgctgagaacgggcaacaactttgaattcagctacaacttcgaggac |
| 1261 | gtgcccttccacagcagctacgcgcacagccagagcctggaccggctgatgaaccctctc |
| 1321 | atcgaccagtacttgtactacctgtcccggactcaaagcacgggcggtactgcaggaact |
| 1381 | cagcagttgctattttctcaggccgggcctaacaacatgtcggctcaggccaagaactgg |
| 1441 | ctacccggtccctgctaccggcagcaacgcgtctccacgacactgtcgcagaacaacaac |
| 1501 | agcaactttgcctggacgggtgccaccaagtatcatctgaatggcagagactctctggtg |
| 1561 | aatcctggcgttgccatggctacccacaaggacgacgaagagcgattttttccatccagc |
| 1621 | ggagtcttaatgtttgggaaacagggagctggaaaagacaacgtggactatagcagcgtg |
| 1681 | atgctaaccagcgaggaagaaataaagaccaccaacccagtggccacagaacagtacggc |
| 1741 | gtggtggccgataacctgcaacagcaaaacgccgctcctattgtaggggccgtcaatagt |
| 1801 | caaggagccttacctggcatggtgtggcagaaccgggacgtgtacctgcagggtcccatc |
| 1861 | tgggccaagattcctcatacggacggcaactttcatccctcgccgctgatgggaggcttt |
| 1921 | ggactgaagcatccgcctcctcagatcctgattaaaaacacacctgttcccgcggatcct |
| 1981 | ccgaccaccttcaatcaggccaagctggcttctttcatcacgcagtacagtaccggccag |
| 2041 | gtcagcgtggagatcgagtgggagctgcagaaggagaacagcaaacgctggaacccagag |
| 2101 | attcagtacacttccaactactacaaatctacaaatgtggactttgctgtcaatactgag |
| 2161 | ggtacttattccgagcctcgccccattggcacccgttacctcacccgtaatctg |

RHM15-1, 15-2, 15-3, 15-4, 1-5 and 15-6 variants also had an alanine, a leucine, a proline, and an asparagine substitution at amino acid positions 195, 199, 201 and 202, respectively, of Rh74 VP1 capsid. In addition, these variants had multiple arginine substitutions of lysine at various positions.

The RHM15-1 variant VP1 capsid amino acid sequence is as follows (SEQ ID NO:6):

| | |
|---|---|
| 1 | maadgylpdwlednlsegirewwdlkpgapkpkanqqrqdngrglvlpgyrylgpfngld |
| 61 | kgepvnaadaaalehdraydgglgagdnpylrynhadaefgerlgedtsfggnlgravfg |
| 121 | akkrvleplglvespvrtapgkkrpvepspgrspdsstgigkkgggparkrinfggtgds |
| 181 | esvpdpgpigeppaapsgvgpntmaagggapmadnnegadgvgsssgnwhcdstwlgdry |
| 241 | ittstrtwalptynnhlyrgisngtsggstndntyfgystpwgyfdfnrfhchfsprdwg |
| 301 | rlinnnwgfrpkrinfklfnigvkevtgnegtrtiannitstigvftdseyglpyvlgsa |
| 361 | hggclppfpadvfmipgygyltlnngsgavgrssfycleyfpsgmlrtgnnfefsynfed |
| 421 | vpfhssyahsgsldrlmnplidgylyylsrtgstggtagtggllfsgagpnnmsagaknw |
| 481 | lpgpcyrggrvsttlsgnnnsnfawtgatkyhlngrdslvnpgvamathrddeerffpss |
| 541 | gvlmfgrggagrdnvdyssvmltseeeirttnpvategygvvadnlgggn aapivgavns |
| 601 | ggalpgmvwgnrdvylggpiwakiphtdgnfhpsplmggfglkhpppgilikntpvpadp |
| 661 | pttfngaklasfitgystggvsveiewelgkenskrwnpeigytsnyykstnvdfavnte |
| 721 | gtyseprpigtryltrnl |

The RHM15-1 variant VP1 capsid nucleic acid sequence is as follows (SEQ ID NO:12):

| | |
|---|---|
| 1 | atggctgccgatggttatcttccagattggctcgaggacaacctctctgagggcattcgc |
| 61 | gagtggtgggacctgaaacctggagccccgaaacccaaagccaaccagcaaaggcaggac |
| 121 | aacggccggggtctggtgcttcctggctacaggtacctcggacccttcaacggactcgac |
| 181 | aagggggagcccgtcaacgcggcggacgcagcggccctcgagcacgacagggcctacgac |
| 241 | cagcagctccaagcgggtgacaatccgtacctgcggtataatcacgccgacgccgagttt |
| 301 | caggagcgtctgcaagaagatacgtcttttgggggcaacctcgggcgcgcagtcttccag |
| 361 | gccaaaaagcgggttctcgaacctctgggcctggttgaatcgccggttaggacggctcct |
| 421 | ggaaagaagagaccggtagagccatcaccccagcgctctccagactcctctacgggcatc |
| 481 | ggcaagaaaggccagcagcccgcaagaaagagactcaattttgggcagactggcgactca |
| 541 | gagtcagtccccgaccctcaaccaatcggagaaccaccagcagccccctctggtgtggga |
| 601 | cctaatacaatggctgcaggcggtggcgctccaatggcagacaataacgaaggcgccgac |
| 661 | ggagtgggtagttcctcaggaaattggcattgcgattccacatggctgggcgacagagtc |
| 721 | atcaccaccagcacccgcacctgggccctgcccacctacaacaaccacctctacaggcaa |
| 781 | atctccaacgggacctcgggaggaagcaccaacgacaacacctacttcggctacagcacc |
| 841 | ccctgggggtattttgacttcaacagattccactgccacttttcaccacgtgactggcag |
| 901 | cgactcatcaacaacaactggggattccggcccaagaggctcaacttcaagctcttcaac |
| 961 | atccaagtcaaggaggtcacgcagaatgaaggcaccagga ccatcgccaa taaccttacc |
| 1021 | agcacgattcaggtctttacggactcggaataccagctcc cgtacgtgct cggctcggcg |
| 1081 | caccagggctgcctgcctccgttcccggcggacgtcttca tgattcctca gtacgggtac |
| 1141 | ctgactctgaacaatggcagtcaggctgtgggccggtcgt ccttctactg cctggagtac |
| 1201 | tttccttctcaaatgctgagaacgggcaacaactttgaat tcagctacaa cttcgaggac |
| 1261 | gtgcccttccacagcagctacgcgcacagccagagcctgg accggctgat gaaccctctc |
| 1321 | atcgaccagtacttgtactacctgtcccggactcaaagca cgggcggtac tgcaggaact |
| 1381 | cagcagttgctattttctcaggccgggcctaacaacatgt cggctcaggc caagaactgg |
| 1441 | ctacccggtccctgctaccggcagcaacgcgtctccacga cactgtcgca gaacaacaac |
| 1501 | agcaactttgcctggacgggtgccaccaagtatcatctga atggcagaga ctctctggtg |
| 1561 | aatcctggcgttgccatggctacccacagggacgacgaag agcgattttt tccatccagc |
| 1621 | ggagtcttaatgtttgggagacagggagctggaagagaca acgtggacta tagcagcgtg |
| 1681 | atgctaaccagcgaggaagaaataaggaccaccaacccag tggccacaga acagtacggc |
| 1741 | gtggtggccgataacctgcaacagcaaaacgccgctccta ttgtaggggc cgtcaatagt |
| 1801 | caaggagccttacctggcatggtgtggcagaaccgggacg tgtacctgca gggtcccatc |
| 1861 | tgggccaagattcctcatacggacggcaactttcatccct cgccgctgat gggaggcttt |
| 1921 | ggactgaagcatccgcctcctcagatcctgattaaaaaca cacctgttcc cgcggatcct |
| 1981 | ccgaccaccttcaatcaggccaagctggcttctttcatca cgcagtacag taccggccag |
| 2041 | gtcagcgtggagatcgagtgggagctgcagaaggagaaca gcaaacgctg gaacccagag |
| 2101 | attcagtacacttccaactactacaaatctacaaatgtgg actttgctgt caatactgag |
| 2161 | ggtacttattccgagcctcgccccattggcacccgttacc tcacccgtaa tctgtaa |

The RHM15-2 variant VP1 capsid amino acid sequence is as follows (SEQ ID NO:7):

| | |
|---|---|
| 1 | maadgylpdwlednlsegirewwdlkpgapkpkanqqrqd ngrglvlpgy rylgpfngld |
| 61 | kgepvnaadaaalehdraydqqlqagdnpylrynhadaef qerlqedtsf ggnlgravfq |
| 121 | akkrvleplglvespvrtapgkkrpvepspqrspdsstgi gkrgqqpark rinfgqtgds |
| 181 | esvpdpqpigeppaapsgvgpntmaagggapmadnnegad gvgsssgnwh cdstwlgdry |
| 241 | ittstrtwalptynnhlyrqisngtsggstndntyfgyst pwgyfdfnrf hchfsprdwq |
| 301 | rlinnnwgfrpkrinfklfniqvkevtqnegtrtiannit stiqvftdse yqlpyvlgsa |
| 361 | hqgclppfpadvfmipqygyltlnngsqavgrssfycley fpsqmlrtgn nfefsynfed |
| 421 | vpfhssyahsqsldrlmnplidqylyylsrtqstggtagt qqllfsqagp nnmsaqaknw |
| 481 | 1pgpcyrqqrvsttlsqnnnsnfawtgatkyhlngrdslv npgvamathr ddeerffpss |
| 541 | gvlmfgkqgagrdnvdyssvmltseeeirttnpvateqyg vvadnlqqqn aapivgavns |
| 601 | qgalpgmvwqnrdvylqgpiwakiphtdgnfhpsplmggf glkhpppqil ikntpvpadp |
| 661 | pttfnqaklasfitqystgqvsveiewelqkenskrwnpe iqytsnyyks tnvdfavnte |
| 721 | gtyseprpigtryltrnl |

The RHM15-2 variant VP1 capsid nucleic acid sequence is as follows (SEQ ID NO:13):

| | |
|---|---|
| 1 | atggctgccgatggttatcttccagattggctcgaggaca acctctctga gggcattcgc |
| 61 | gagtggtgggacctgaaacctggagccccgaaacccaaag ccaaccagca aaggcaggac |
| 121 | aacggccggggtctggtgcttcctggctacaggtacctcg gacccttcaa cggactcgac |
| 181 | aagggggagcccgtcaacgcggcggacgcagcggccctcg agcacgacag ggcctacgac |
| 241 | cagcagctccaagcgggtgacaatccgtacctgcggtata atcacgccga cgccgagttt |
| 301 | caggagcgtctgcaagaagatacgtcttttgggggcaacc tcgggcgcgc agtcttccag |
| 361 | gccaaaaagcgggttctcgaacctctgggcctggttgaat cgccggttag gacggctcct |
| 421 | ggaaagaagagaccggtagagccatcaccccagcgctctc cagactcctc tacgggcatc |
| 481 | ggcaagagaggccagcagcccgcaagaaagagactcaatt ttgggcagac tggcgactca |
| 541 | gagtcagtccccgaccctcaaccaatcggagaaccaccag cagccccctc tggtgtggga |
| 601 | cctaatacaatggctgcaggcggtggcgctccaatggcag acaataacga aggcgccgac |
| 661 | ggagtgggtagttcctcaggaaattggcattgcgattcca catggctggg cgacagagtc |
| 721 | atcaccaccagcacccgcacctgggccctgcccacctaca acaaccacct ctacaggcaa |
| 781 | atctccaacgggacctcgggaggaagcaccaacgacaaca cctacttcgg ctacagcacc |
| 841 | ccctgggggtattttgacttcaacagattccactgccact tttcaccacg tgactggcag |
| 901 | cgactcatcaacaacaactggggattccggcccaagaggc tcaacttcaa gctcttcaac |
| 961 | atccaagtcaaggaggtcacgcagaatgaaggcaccagga ccatcgccaa taaccttacc |
| 1021 | agcacgattcaggtctttacggactcggaataccagctcc cgtacgtgct cggctcggcg |
| 1081 | caccagggctgcctgcctccgttcccggcggacgtcttca tgattcctca gtacgggtac |
| 1141 | ctgactctgaacaatggcagtcaggctgtgggccggtcgt ccttctactg cctggagtac |
| 1201 | tttccttctcaaatgctgagaacgggcaacaactttgaat tcagctacaa cttcgaggac |
| 1261 | gtgcccttccacagcagctacgcgcacagccagagcctgg accggctgat gaaccctctc |
| 1321 | atcgaccagtacttgtactacctgtcccggactcaaagca cgggcggtac tgcaggaact |
| 1381 | cagcagttgctattttctcaggccgggcctaacaacatgt cggctcaggc caagaactgg |
| 1441 | ctacccggtccctgctaccggcagcaacgcgtctccacga cactgtcgca gaacaacaac |
| 1501 | agcaactttgcctggacgggtgccaccaagtatcatctga atggcagaga ctctctggtg |
| 1561 | aatcctggcgttgccatggctacccacagggacgacgaag agcgattttt tccatccagc |
| 1621 | ggagtcttaatgtttgggaaacagggagctggaagagaca acgtggacta tagcagcgtg |
| 1681 | atgctaaccagcgaggaagaaataaggaccaccaacccag tggccacaga acagtacggc |
| 1741 | gtggtggccgataacctgcaacagcaaaacgccgctccta ttgtaggggc cgtcaatagt |
| 1801 | caaggagccttacctggcatggtgtggcagaaccgggacg tgtacctgca gggtcccatc |
| 1861 | tgggccaagattcctcatacggacggcaactttcatccct cgccgctgat gggaggcttt |
| 1921 | ggactgaagcatccgcctcctcagatcctgattaaaaaca cacctgttcc cgcggatcct |
| 1981 | ccgaccaccttcaatcaggccaagctggcttctttcatca cgcagtacag taccggccag |
| 2041 | gtcagcgtggagatcgagtgggagctgcagaaggagaaca gcaaacgctg gaacccagag |
| 2101 | attcagtacacttccaactactacaaatctacaaatgtgg actttgctgt caatactgag |
| 2161 | ggtacttattccgagcctcgccccattggcacccgttacc tcacccgtaa tctgtaa |

The RHM15-3/RHM15-5 variant VP1 capsid amino acid sequence is as follows (SEQ ID NO:8)

| | |
|---|---|
| 1 | maadgylpdwlednlsegir ewwdlkpgapkpkanqqrqd ngrglvlpgy rylgpfngld |
| 61 | kgepvnaadaaalehdraydqqlqagdnpylrynhadaef qerlqedtsf ggnlgravfq |
| 121 | akkrvleplglvespvrtapgkkrpvepspqrspdsstgi gkrgqqpakk rinfgqtgds |
| 181 | esvpdpqpigeppaapsgvgpntmaagggapmadnnegad gvgsssgnwh cdstwlgdry |
| 241 | ittstrtwalptynnhlyrqisngtsggstndntyfgyst pwgyfdfnrf hchfsprdwq |
| 301 | rlinnnwgfrpkrinfklfniqvkevtqnegtrtiannit stiqvftdse yqlpyvlgsa |
| 361 | hqgclppfpadvfmipqygyltlnngsqavgrssfycley fpsqmlrtgn nfefsynfed |
| 421 | vpfhssyahsqsldrlmnplidqylyylsrtqstggtagt qqllfsqagp nnmsaqaknw |
| 481 | lpgpcyrqqrvsttlsqnnnsnfawtgatkyhlngrdslv npgvamathr ddeerffpss |
| 541 | gvlmfgrqgagrdnvdyssvmltseeeirttnpvateqyg vvadnlqqqn aapivgavns |
| 601 | qgalpgmvwqnrdvylqgpiwakiphtdgnfhpsplmggf glkhpppqil ikntpvpadp |
| 661 | pttfnqaklasfitqystgqvsveiewelqkenskrwnpe iqytsnyyks tnvdfavnte |
| 721 | gtyseprpigtryltrnl |

The RHM15-3/RHM15-5 variant VP1 capsid nucleic acid sequence is as follows (SEQ ID NO:14):

| | |
|---|---|
| 1 | atggctgccgatggttatcttccagattggctcgaggaca acctctctga gggcattcgc |
| 61 | gagtggtgggacctgaaacctggagccccgaaacccaaag ccaaccagca aaggcaggac |
| 121 | aacggccggggtctggtgcttcctggctacaggtacctcg gacccttcaa cggactcgac |
| 181 | aagggggagcccgtcaacgcggcggacgcagcggccctcg agcacgacag ggcctacgac |
| 241 | cagcagctccaagcgggtgacaatccgtacctgcggtata atcacgccga cgccgagttt |
| 301 | caggagcgtctgcaagaagatacgtcttttgggggcaacc tcgggcgcgc agtcttccag |
| 361 | gccaaaaagcgggttctcgaacctctgggcctggttgaat cgccggttag gacggctcct |
| 421 | ggaaagaagagaccggtagagccatcaccccagcgctctc cagactcctc tacgggcatc |
| 481 | ggcaagagaggccagcagcccgcaaaaaagagactcaatt ttgggcagac tggcgactca |
| 541 | gagtcagtccccgaccctcaaccaatcggagaaccaccag cagccccctc tggtgtggga |
| 601 | cctaatacaatggctgcaggcggtggcgctccaatggcag acaataacga aggcgccgac |
| 661 | ggagtgggtagttcctcaggaaattggcattgcgattcca catggctggg cgacagagtc |
| 721 | atcaccaccagcacccgcacctgggccctgcccacctaca acaaccacct ctacaggcaa |
| 781 | atctccaacgggacctcgggaggaagcaccaacgacaaca cctacttcgg ctacagcacc |
| 841 | ccctgggggtattttgacttcaacagattccactgccact tttcaccacg tgactggcag |
| 901 | cgactcatcaacaacaactggggattccggcccaagaggc tcaacttcaa gctcttcaac |
| 961 | atccaagtcaaggaggtcacgcagaatgaaggcaccagga ccatcgccaa taaccttacc |
| 1021 | agcacgattcaggtctttacggactcggaataccagctcc cgtacgtgct cggctcggcg |
| 1081 | caccagggctgcctgcctccgttcccggcggacgtcttca tgattcctca gtacgggtac |
| 1141 | ctgactctgaacaatggcagtcaggctgtgggccggtcgt ccttctactg cctggagtac |
| 1201 | tttccttctcaaatgctgagaacgggcaacaactttgaat tcagctacaa cttcgaggac |
| 1261 | gtgcccttccacagcagctacgcgcacagccagagcctgg accggctgat gaaccctctc |
| 1321 | atcgaccagtacttgtactacctgtcccggactcaaagca cgggcggtac tgcaggaact |
| 1381 | cagcagttgctattttctcaggccgggcctaacaacatgt cggctcaggc caagaactgg |
| 1441 | ctacccggtccctgctaccggcagcaacgcgtctccacga cactgtcgca gaacaacaac |
| 1501 | agcaactttgcctggacgggtgccaccaagtatcatctga atggcagaga ctctctggtg |
| 1561 | aatcctggcgttgccatggctacccacagggacgacgaag agcgattttt tccatccagc |
| 1621 | ggagtcttaatgtttgggagacagggagctggaagagaca acgtggacta tagcagcgtg |
| 1681 | atgctaaccagcgaggaagaaataaggaccaccaacccag tggccacaga acagtacggc |
| 1741 | gtggtggccgataacctgcaacagcaaaacgccgctccta ttgtaggggc cgtcaatagt |
| 1801 | caaggagccttacctggcatggtgtggcagaaccgggacg tgtacctgca gggtcccatc |
| 1861 | tgggccaagattcctcatacggacggcaactttcatccct cgccgctgat gggaggcttt |
| 1921 | ggactgaagcatccgcctcctcagatcctgattaaaaaca cacctgttcc cgcggatcct |
| 1981 | ccgaccaccttcaatcaggccaagctggcttctttcatca cgcagtacag taccggccag |
| 2041 | gtcagcgtggagatcgagtgggagctgcagaaggagaaca gcaaacgctg gaacccagag |
| 2101 | attcagtacacttccaactactacaaatctacaaatgtgg actttgctgt caatactgag |
| 2161 | ggtacttattccgagcctcgccccattggcacccgttacc tcacccgtaa tctgtaa |

The RHM15-4 variant VP1 capsid amino acid sequence is as follows (SEQ ID NO:9):

| | |
|---|---|
| 1 | maadgylpdwlednlsegirewwdlkpgap kpkanqqrqd ngrglvlpgy rylgpfngld |
| 61 | kgepvnaadaaalehdraydqqlqagdnpy lrynhadaef qerlqedtsf ggnlgravfq |
| 121 | akkrvleplglvespvrtapgkkrpvepsp qrspdsstgi gkrgqqpakk rinfgqtgds |
| 181 | esvpdpqpigeppaapsgvgpntmaaggga pmadnnegad gvgsssgnwh cdstwlgdry |
| 241 | ittstrtwalptynnhlyrqisngtsggst ndntyfgyst pwgyfdfnrf hchfsprdwq |
| 301 | rlinnnwgfrpkrinfklfniqvkevtqne gtrtiannit stiqvftdse yqlpyvlgsa |
| 361 | hqgclppfpadvfmipqygyltlnngsqav grssfycley fpsqmlrtgn nfefsynfed |
| 421 | vpfhssyahsqsldrlmnplidqylyylsr tqstggtagt qqllfsqagp nnmsaqaknw |
| 481 | 1pgpcyrqqrvsttlsqnnnsnfawtgatk yhlngrdslv npgvamathr ddeerffpss |
| 541 | gvlmfgkqgagrdnvdyssvmltseeeirt tnpvateqyg vvadnlqqqn aapivgavns |
| 601 | qgalpgmvwqnrdvylqgpiwakiphtdgn fhpsplmggf glkhpppqil ikntpvpadp |
| 661 | pttfnqarlasfitqystgqvsveiewelq kenskrwnpe iqytsnyyks tnvdfavnte |
| 721 | gtyseprpigtryltrnl |

The RHM15-4 variant VP1 capsid nucleic acid sequence is as follows (SEQ ID NO: 15):

| | |
|---|---|
| 1 | atggctgccgatggttatcttccagattggctcgaggaca acctctctga gggcattcgc |
| 61 | gagtggtgggacctgaaacctggagccccgaaacccaaag ccaaccagca aaggcaggac |
| 121 | aacggccggggtctggtgcttcctggctacaggtacctcg gacccttcaa cggactcgac |
| 181 | aagggggagcccgtcaacgcggcggacgcagcggccctcg agcacgacag ggcctacgac |
| 241 | cagcagctccaagcgggtgacaatccgtacctgcggtata atcacgccga cgccgagttt |
| 301 | caggagcgtctgcaagaagatacgtcttttgggggcaacc tcgggcgcgc agtcttccag |
| 361 | gccaaaaagcgggttctcgaacctctgggcctggttgaat cgccggttag gacggctcct |
| 421 | ggaaagaagagaccggtagagccatcaccccagcgctctc cagactcctc tacgggcatc |
| 481 | ggcaagagaggccagcagcccgcaaaaaagagactcaatt ttgggcagac tggcgactca |
| 541 | gagtcagtccccgaccctcaaccaatcggagaaccaccag cagccccctc tggtgtggga |
| 601 | cctaatacaatggctgcaggcggtggcgctccaatggcag acaataacga aggcgccgac |
| 661 | ggagtgggtagttcctcaggaaattggcattgcgattcca catggctggg cgacagagtc |
| 721 | atcaccaccagcacccgcacctgggccctgcccacctaca acaaccacct ctacaggcaa |
| 781 | atctccaacgggacctcgggaggaagcaccaacgacaaca cctacttcgg ctacagcacc |
| 841 | ccctgggggtattttgacttcaacagattccactgccact tttcaccacg tgactggcag |
| 901 | cgactcatcaacaacaactggggattccggcccaagaggc tcaacttcaa gctcttcaac |
| 961 | atccaagtcaaggaggtcacgcagaatgaaggcaccagga ccatcgccaa taaccttacc |
| 1021 | agcacgattcaggtctttacggactcggaataccagctcc cgtacgtgct cggctcggcg |
| 1081 | caccagggctgcctgcctccgttcccggcggacgtcttca tgattcctca gtacgggtac |
| 1141 | ctgactctgaacaatggcagtcaggctgtgggccggtcgt ccttctactg cctggagtac |
| 1201 | tttccttctcaaatgctgagaacgggcaacaactttgaat tcagctacaa cttcgaggac |
| 1261 | gtgcccttccacagcagctacgcgcacagccagagcctgg accggctgat gaaccctctc |
| 1321 | atcgaccagtacttgtactacctgtcccggactcaaagca cgggcggtac tgcaggaact |
| 1381 | cagcagttgctattttctcaggccgggcctaacaacatgt cggctcaggc caagaactgg |
| 1441 | ctacccggtccctgctaccggcagcaacgcgtctccacga cactgtcgca gaacaacaac |
| 1501 | agcaactttgcctggacgggtgccaccaagtatcatctga atggcagaga ctctctggtg |
| 1561 | aatcctggcgttgccatggctacccacagggacgacgaag agcgattttt tccatccagc |
| 1621 | ggagtcttaatgtttgggaaacagggagctggaagagaca acgtggacta tagcagcgtg |
| 1681 | atgctaaccagcgaggaagaaataaggaccaccaacccag tggccacaga acagtacggc |
| 1741 | gtggtggccgataacctgcaacagcaaaacgccgctccta ttgtaggggc cgtcaatagt |
| 1801 | caaggagccttacctggcatggtgtggcagaaccgggacg tgtacctgca gggtcccatc |
| 1861 | tgggccaagattcctcatacggacggcaactttcatccct cgccgctgat gggaggcttt |
| 1921 | ggactgaagcatccgcctcctcagatcctgattaaaaaca cacctgttcc cgcggatcct |
| 1981 | ccgaccaccttcaatcaggccaggctggcttctttcatca cgcagtacag taccggccag |
| 2041 | gtcagcgtggagatcgagtgggagctgcagaaggagaaca gcaaacgctg gaacccagag |
| 2101 | attcagtacacttccaactactacaaatctacaaatgtgg actttgctgt caatactgag |
| 2161 | ggtacttattccgagcctcgccccattggcacccgttacc tcacccgtaa tctgtaa |

The RHM15-6 variant VP1 capsid amino acid sequence is as follows (SEQ ID NO:10):

| | |
|---|---|
| 1 | maadgylpdwlednlsegirewwdlkpgapkpkanqqrqdngrglvlpgy rylgpfngld |
| 61 | kgepvnaadaaalehdraydqqlqagdnpylrynhadaefgerlqedtsf ggnlgravfq |
| 121 | akkrvleplglvespvrtapgkkrpvepspqrspdsstgigkkgqqpakk rinfgqtgds |
| 181 | esvpdpqpigeppaapsgvgpntmaagggapmadnnegadgvgsssgnwh cdstwlgdry |
| 241 | ittstrtwalptynnhlyrgisngtsggstndntyfgystpwgyfdfnrf hchfsprdwq |
| 301 | rlinnnwgfrpkrinfklfniqvkevtqnegtrtiannitstiqvftdse yqlpyvlgsa |
| 361 | hqgclppfpadvfmipqygyltlnngsgavgrssfycleyfpsqm1rtgn nfefsynfed |
| 421 | vpfhssyahsqsldrlmnplidqylyylsrtqstggtagtqqllfsqagp nnmsaqaknw |
| 481 | 1pgpcyrqqrvsttlsqnnnsnfawtgatkyhlngrdslvnpgvamathr ddeerffpss |
| 541 | gvlmfgrqgagrdnvdyssvmltseeeirttnpvateqygvvadnlqqqn aapivgavns |
| 601 | qgalpgmvwqnrdvylqgpiwakiphtdgnfhpsplmggfglkhpppgil ikntpvpadp |
| 661 | pttfnqarlasfitqystgqvsveiewelqkenskrwnpeiqytsnyyks tnvdfavnte |
| 721 | gtyseprpigtryltrnl |

The RHM15-6 variant VP1 capsid nucleic acid sequence is as follows (SEQ ID NO: 16):

| | |
|---|---|
| 1 | atggctgccgatggttatcttccagattggctcgaggaca acctctctga gggcattcgc |
| 61 | gagtggtgggacctgaaacctggagccccgaaacccaaag ccaaccagca aaggcaggac |
| 121 | aacggccggggtctggtgcttcctggctacaggtacctcg gacccttcaa cggactcgac |
| 181 | aagggggagcccgtcaacgcggcggacgcagcggccctcg agcacgacag ggcctacgac |
| 241 | cagcagctccaagcgggtgacaatccgtacctgcggtata atcacgccga cgccgagttt |
| 301 | caggagcgtctgcaagaagatacgtcttttgggggcaacc tcgggcgcgc agtcttccag |
| 361 | gccaaaaagcgggttctcgaacctctgggcctggttgaat cgccggttag gacggctcct |
| 421 | ggaaagaagagaccggtagagccatcaccccagcgctctc cagactcctc tacgggcatc |
| 481 | ggcaagaaaggccagcagcccgcaaaaaagagactcaatt ttgggcagac tggcgactca |
| 541 | gagtcagtccccgaccctcaaccaatcggagaaccaccag cagccccctc tggtgtggga |
| 601 | cctaatacaatggctgcaggcggtggcgctccaatggcag acaataacga aggcgccgac |
| 661 | ggagtgggtagttcctcaggaaattggcattgcgattcca catggctggg cgacagagtc |
| 721 | atcaccaccagcacccgcacctgggccctgcccacctaca acaaccacct ctacaggcaa |
| 781 | atctccaacgggacctcgggaggaagcaccaacgacaaca cctacttcgg ctacagcacc |
| 841 | ccctgggggtattttgacttcaacagattccactgccact tttcaccacg tgactggcag |
| 901 | cgactcatcaacaacaactggggattccggcccaagaggc tcaacttcaa gctcttcaac |
| 961 | atccaagtcaaggaggtcacgcagaatgaaggcaccagga ccatcgccaa taaccttacc |
| 1021 | agcacgattcaggtctttacggactcggaataccagctcc cgtacgtgct cggctcggcg |
| 1081 | caccagggctgcctgcctccgttcccggcggacgtcttca tgattcctca gtacgggtac |
| 1141 | ctgactctgaacaatggcagtcaggctgtgggccggtcgt ccttctactg cctggagtac |
| 1201 | tttccttctcaaatgctgagaacgggcaacaactttgaat tcagctacaa cttcgaggac |
| 1261 | gtgcccttccacagcagctacgcgcacagccagagcctgg accggctgat gaaccctctc |
| 1321 | atcgaccagtacttgtactacctgtcccggactcaaagca cgggcggtac tgcaggaact |
| 1381 | cagcagttgctattttctcaggccgggcctaacaacatgt cggctcaggc caagaactgg |
| 1441 | ctacccggtccctgctaccggcagcaacgcgtctccacga cactgtcgca gaacaacaac |
| 1501 | agcaactttgcctggacgggtgccaccaagtatcatctga atggcagaga ctctctggtg |
| 1561 | aatcctggcgttgccatggctacccacagggacgacgaag agcgattttt tccatccagc |
| 1621 | ggagtcttaatgtttgggagacagggagctggaagagaca acgtggacta tagcagcgtg |
| 1681 | atgctaaccagcgaggaagaaataaggaccaccaacccag tggccacaga acagtacggc |
| 1741 | gtggtggccgataacctgcaacagcaaaacgccgctccta ttgtaggggc cgtcaatagt |
| 1801 | caaggagccttacctggcatggtgtggcagaaccgggacg tgtacctgca gggtcccatc |
| 1861 | tgggccaagattcctcatacggacggcaactttcatccct cgccgctgat gggaggcttt |
| 1921 | ggactgaagcatccgcctcctcagatcctgattaaaaaca cacctgttcc cgcggatcct |
| 1981 | ccgaccaccttcaatcaggccaggctggcttctttcatca cgcagtacag taccggccag |
| 2041 | gtcagcgtggagatcgagtgggagctgcagaaggagaaca gcaaacgctg gaacccagag |
| 2101 | attcagtacacttccaactactacaaatctacaaatgtgg actttgctgt caatactgag |
| 2161 | ggtacttattccgagcctcgccccattggcacccgttacc tcacccgtaa tctgtaa |

### Example 8

### This Example Includes a Description of Human Factor IX Expression Studies Using the Rh74 Capsid Variants Compared to Rh74 and AAV8.

In brief, Rh74 capsid variants were used to package an AAV human factor IX expression vector, the AAV particles used to infect mice, and expression levels of factor IX determined in the animals (plasma). The capsid variants had the following amino acid substitutions at the indicated positions:

TABLE 3

| | |
|---|---|
| RHM4_1 | G195A-L199V- S201P-G202N |
| RHM15_1 | G195A-L199V-S201P-G202N |
| | K(137/259/333/530/552/569/38/51/77/169/547)R |
| RHM15_2 | G195A-L199V- S201P-G202N |
| | K(137/259/333/530/552/569/38/51/77/163/169)R |
| RHM15_3 | G195A-L199V- S201P-G202N |
| | K(137/259/333/530/552/569/38/51/77/163/547)R |
| RHM15_4 | G195A-L199V- S201P-G202N |
| | K(137/259/333/530/552/569/38/51/77/163/668)R |
| RHM15_5 | G195A-L199V- S201P-G202N |
| | K(137/259/333/530/552/569/38/51/77/547/163)R |
| RHM15_6 | G195A-L199V- S201P-G202N |
| | K(137/259/333/530/552/569/38/51/77/547/688)R |

FIG. 5 shows plasma human factor IX expression levels in treated animals after 2 weeks. As illustrated, AAV human factor IX expression vector encapsidated by RHM4-1 variant Capsid provided the highest level of expression, and was substantially greater than expression produced by Rh74 encapsidated AAV human factor IX expression vector, and AAV8 encapsidated AAV human factor IX expression vector.

### Example 9

This Example Includes a Description of Data Showing that AAVrh74 Variant RHM4-1 Vector Expressing Human Factor IX (FIX) Under the Control of a Liver-Specific Promoter Administered to Cynomolgus Macaques Led to Production Amounts of FIX in Animals, and at Higher Levels than AAV8 Vector Administered at the Same Amount.

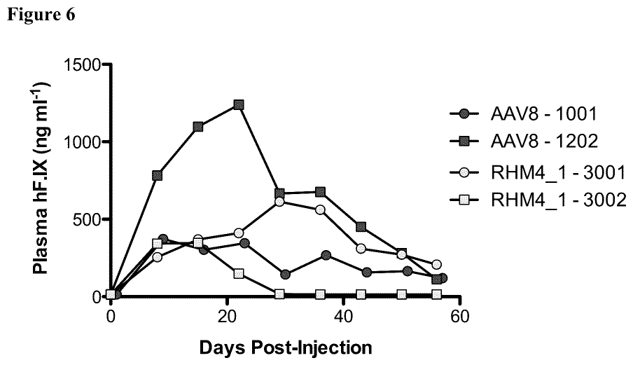

Cynomolgus monkeys were prescreened for neutralizing AAV antibodies, and animals with pre-treatment titers of <1:1 were selected to ensure that transduction was successful. The monkeys were then infused with either AAV8 or AAV-Rh74 variant RHM4-1 vectors expressing a human factor IX transgene at a dose of 3×10¹² vg/kg. Human FIX transgene product (protein) plasma levels in the non-human primates were determined by ELISA weekly throughout the duration of the study and are illustrated in FIG. 6.

One of the animals receiving AAV-RHM4-1-FIX developed an inhibitor against the human factor IX transgene product, which is a well-documented phenomenon that occurs in approximately 20% of macaques treated with a human FIX vector owing to small amino acid differences between the human and macaque proteins. The loss of expression in one animal treated with RHM4-1 was due to the development of an antibody response against the human transgene. The second RHM4-1-treated animal expressed FIX levels around 2-fold higher compared to the AAV8-treated macaques.

1-56. (canceled)

57. A recombinant AAV particle comprising an AAV capsid sequence which comprises RHM4-1 (SEQ ID NO:5), wherein the particle encapsidates an AAV vector genome that comprises a heterologous polynucleotide sequence that is transcribed into an inhibitory nucleic acid that inhibits expression of huntingtin (HTT) gene.

58. A recombinant AAV particle comprising an AAV capsid sequence which comprises RHM4-1 (SEQ ID NO:5), wherein the particle encapsidates an AAV vector genome that comprises a heterologous polynucleotide sequence that is transcribed into an inhibitory nucleic acid that inhibits expression of a gene involved in pathogenesis of Parkinson's disease.

59. The recombinant AAV particle of claim 57 or 58, wherein the heterologous polynucleotide sequence is operably linked to an expression control element conferring transcription of said heterologous polynucleotide sequence.

60. The recombinant AAV particle of claim 57 or 58, wherein the inhibitory nucleic acid comprises micro-RNA (miRNA), siRNA, shRNA, trans-splicing RNA, antisense RNA or triplex forming RNA.

61. The recombinant AAV particle of claim 57, wherein the inhibitory nucleic acid binds to a pathogenic HTT gene, or a transcript of a pathogenic HTT gene.

62. The recombinant AAV particle of claim 59, wherein the expression control element comprises a constitutive or regulatable control element.

63. The recombinant AAV particle of claim 59, wherein the expression control element comprises a tissue-specific expression control element or promoter.

64. A pharmaceutical composition comprising the recombinant AAV particle of claim 57 or 58.

65. The pharmaceutical composition of claim 64, further comprising AAV empty capsid.

66. A method of delivering a recombinant AAV particle to a mammal, comprising: (a) providing the recombinant AAV particle claim 57 or 58; and (b) administering the recombinant AAV particle to said mammal.

67. The method of claim 66, wherein the AAV vector is delivered intra-cranially, intravenously or intraarterially.

68. The method of claim 66, wherein the mammal is human.

69. The method of claim 66, wherein the mammal is sero-positive for any AAV serotype.

70. The method of claim 66, wherein the mammal is sero-negative for AAV-Rh74 or RHM4-1 (SEQ ID NO:5).

71. The method of claim 66, further comprising administering AAV empty capsid.

72. The method of claim 66, further comprising administering AAV empty capsid RHM4-1 (SEQ ID NO:5).


SEQUENCE LISTING (16 sequences)

SEQ ID NO: 1 - protein, 738 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - DNA, 2217 bp, Adeno-associated virus
  source 1..2217: mol_type = other DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 3 - protein, 601 aa, Adeno-associated virus
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 4 - protein, 535 aa, Adeno-associated virus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 5 - protein, 738 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 6 - protein, 738 aa, Adeno-associated virus
MAADGYLPDWLEDNLSEGIREWWDLKPGAPKPKANQQRQDNGRGLVLPGYRYLGPFNGLDKGEPVNAADAAALEHDRAYDQQLQAGDNPYLRYNHADAEFQERLQEDTSFGGNLGRAVFQAKKRVLEPLGLVESPVRTAPGKKRPVEPSPQRSPDSSTGIGKKGQQPARKRLNFGQTGDSESVPDPQPIGEPPAAPSGVGPNTMAAGGGAPMADNNEGADGVGSSSGNWHCDSTWLGDRVITTSTRTWALPTYNNHLYRQISNGTSGGSTNDNTYFGYSTPWGYFDFNRFHCHFSPRDWQRLINNNWGFRPKRLNFKLFNIQVKEVTQNEGTRTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHRDDEERFFPSSGVLMFGRQGAGRDNVDYSSVMLTSEEEIRTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 7 - protein, 738 aa, Adeno-associated virus
MAADGYLPDWLEDNLSEGIREWWDLKPGAPKPKANQQRQDNGRGLVLPGYRYLGPFNGLDKGEPVNAADAAALEHDRAYDQQLQAGDNPYLRYNHADAEFQERLQEDTSFGGNLGRAVFQAKKRVLEPLGLVESPVRTAPGKKRPVEPSPQRSPDSSTGIGKRGQQPARKRLNFGQTGDSESVPDPQPIGEPPAAPSGVGPNTMAAGGGAPMADNNEGADGVGSSSGNWHCDSTWLGDRVITTSTRTWALPTYNNHLYRQISNGTSGGSTNDNTYFGYSTPWGYFDFNRFHCHFSPRDWQRLINNNWGFRPKRLNFKLFNIQVKEVTQNEGTRTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHRDDEERFFPSSGVLMFGKQGAGRDNVDYSSVMLTSEEEIRTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 8 - protein, 738 aa, Adeno-associated virus
MAADGYLPDWLEDNLSEGIREWWDLKPGAPKPKANQQRQDNGRGLVLPGYRYLGPFNGLDKGEPVNAADAAALEHDRAYDQQLQAGDNPYLRYNHADAEFQERLQEDTSFGGNLGRAVFQAKKRVLEPLGLVESPVRTAPGKKRPVEPSPQRSPDSSTGIGKRGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYRQISNGTSGGSTNDNTYFGYSTPWGYFDFNRFHCHFSPRDWQRLINNNWGFRPKRLNFKLFNIQVKEVTQNEGTRTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHRDDEERFFPSSGVLMFGRQGAGRDNVDYSSVMLTSEEEIRTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 9 - protein, 738 aa, Adeno-associated virus
MAADGYLPDWLEDNLSEGIREWWDLKPGAPKPKANQQRQDNGRGLVLPGYRYLGPFNGLDKGEPVNAADAAALEHDRAYDQQLQAGDNPYLRYNHADAEFQERLQEDTSFGGNLGRAVFQAKKRVLEPLGLVESPVRTAPGKKRPVEPSPQRSPDSSTGIGKRGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYRQISNGTSGGSTNDNTYFGYSTPWGYFDFNRFHCHFSPRDWQRLINNNWGFRPKRLNFKLFNIQVKEVTQNEGTRTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHRDDEERFFPSSGVLMFGKQGAGRDNVDYSSVMLTSEEEIRTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQARLASFITQYSTGQVSVEIEWELQKENSKRWNPEIQYTSNYYKSTNVDFAVNTEGTYSEPRPIGTRYLTRNL

SEQ ID NO: 10 - protein, 738 aa, Adeno-associated virus
MAADGYLPDWLEDNLSEGIREWWDLKPGAPKPKANQQRQDNGRGLVLPGYRYLGPFNGLDKGEPVNAADAAALEHDRAYDQQLQAGDNPYLRYNHADAEFQERLQEDTSFGGNLGRAVFQAKKRVLEPLGLVESPVRTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYRQISNGTSGGSTNDNTYFGYSTPWGYFDFNRFHCHFSPRDWQRLINNNWGFRPKRLNFKLFNIQVKEVTQNEGTRTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHRDDEERFFPSSGVLMFGRQGAGRDNVDYSSVMLTSEEEIRTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQARLASFITQYSTGQVSVEIEWELQKENSKRWNPEIQYTSNYYKSTNVDFAVNTEGTYSEPRPIGTRYLTRNL

SEQ ID NO: 11 - DNA, 2214 bp, Adeno-associated virus
  source 1..2214: mol_type = other DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcagccccctctggtgtgggacctaatacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctg

SEQ ID NO: 12 - DNA, 2217 bp, Adeno-associated virus
  source 1..2217: mol_type = other DNA
atggctgccgatggttatcttccagattggctcgaggacaacctctctgagggcattcgcgagtggtgggacctgaaacctggagccccgaaacccaaagccaaccagcaaaggcaggacaacggccggggtctggtgcttcctggctacaggtacctcggacccttcaacggactcgacaagggggagcccgtcaacgcggcggacgcagcggccctcgagcacgacagggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaggacggctcctggaaagaagagaccggtagagccatcaccccagcgctctccagactcctctacgggcatcggcaagaaaggccagcagcccgcaagaaagagactcaattttgggcagactggcgactcagagtcagtccccgaccctcaaccaatcggagaaccaccagcagccccctctggtgtgggacctaatacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaggcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaggaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacagggacgacgaagagcgattttttccatccagcggagtcttaatgtttgggagacagggagctggaagagacaacgtggactatagcagcgtgatgctaaccagcgaggaagaaataaggaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 13 - DNA, 2217 bp, Adeno-associated virus
  source 1..2217: mol_type = other DNA
atggctgccgatggttatcttccagattggctcgaggacaacctctctgagggcattcgcgagtggtgggacctgaaacctggagccccgaaacccaaagccaaccagcaaaggcaggacaacggccggggtctggtgcttcctggctacaggtacctcggacccttcaacggactcgacaagggggagcccgtcaacgcggcggacgcagcggccctcgagcacgacagggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaggacggctcctggaaagaagagaccggtagagccatcaccccagcgctctccagactcctctacgggcatcggcaagagaggccagcagcccgcaagaaagagactcaattttgggcagactggcgactcagagtcagtccccgaccctcaaccaatcggagaaccaccagcagccccctctggtgtgggacctaatacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaggcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaggaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacagggacgacgaagagcgattttttccatccagcggagtcttaatgtttgggaaacagggagctggaagagacaacgtggactatagcagcgtgatgctaaccagcgaggaagaaataaggaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 14 - DNA, 2217 bp, Adeno-associated virus
  source 1..2217: mol_type = other DNA
atggctgccgatggttatcttccagattggctcgaggacaacctctctgagggcattcgcgagtggtgggacctgaaacctggagccccgaaacccaaagccaaccagcaaaggcaggacaacggccggggtctggtgcttcctggctacaggtacctcggacccttcaacggactcgacaagggggagcccgtcaacgcggcggacgcagcggccctcgagcacgacagggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaggacggctcctggaaagaagagaccggtagagccatcaccccagcgctctccagactcctctacgggcatcggcaagagaggccagcagcccgcaaaaaagagactcaattttgggcagactggcgactcagagtcagtccccgaccctcaaccaatcggagaaccaccagcagccccctctggtgtgggacctaatacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaggcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaggaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacagggacgacgaagagcgattttttccatccagcggagtcttaatgtttgggagacagggagctggaagagacaacgtggactatagcagcgtgatgctaaccagcgaggaagaaataaggaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 15 - DNA, 2217 bp, Adeno-associated virus
  source 1..2217: mol_type = other DNA
atggctgccgatggttatcttccagattggctcgaggacaacctctctgagggcattcgcgagtggtgggacctgaaacctggagccccgaaacccaaagccaaccagcaaaggcaggacaacggccggggtctggtgcttcctggctacaggtacctcggacccttcaacggactcgacaagggggagcccgtcaacgcggcggacgcagcggccctcgagcacgacagggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaggacggctcctggaaagaagagaccggtagagccatcaccccagcgctctccagactcctctacgggcatcggcaagagaggccagcagcccgcaaaaaagagactcaattttgggcagactggcgactcagagtcagtccccgaccctcaaccaatcggagaaccaccagcagccccctctggtgtgggacctaatacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaggcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaggaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacagggacgacgaagagcgattttttccatccagcggagtcttaatgtttgggaaacagggagctggaagagacaacgtggactatagcagcgtgatgctaaccagcgaggaagaaataaggaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaggctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 16 - DNA, 2217 bp, Adeno-associated virus
  source 1..2217: mol_type = other DNA
atggctgccgatggttatcttccagattggctcgaggacaacctctctgagggcattcgcgagtggtgggacctgaaacctggagccccgaaacccaaagccaaccagcaaaggcaggacaacggccggggtctggtgcttcctggctacaggtacctcggacccttcaacggactcgacaagggggagcccgtcaacgcggcggacgcagcggccctcgagcacgacagggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaggacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcagccccctctggtgtgggacctaatacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaggcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaggaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacagggacgacgaagagcgattttttccatccagcggagtcttaatgtttgggagacagggagctggaagagacaacgtggactatagcagcgtgatgctaaccagcgaggaagaaataaggaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaggctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa